(19) United States
(12) Patent Application Publication
(10) Pub. No.: US 2018/0321241 A1
(43) Pub. Date: Nov. 8, 2018

(54) COMPOSITIONS AND METHODS FOR CARTILAGE AND BONE REGENERATIVE THERAPY

(71) Applicants: Peter Giannoudis, Leeds (GB); Jehan El-Jawhari, Warsaw, IN (US); Paul D'Antonio, Warsaw, IN (US); William King, Warsaw, IN (US)

(72) Inventors: Peter Giannoudis, Leeds (GB); Jehan El-Jawhari, Warsaw, IN (US); Paul D'Antonio, Warsaw, IN (US); William King, Warsaw, IN (US)

(21) Appl. No.: 15/973,025

(22) Filed: May 7, 2018

Related U.S. Application Data
(60) Provisional application No. 62/502,902, filed on May 8, 2017.

(51) Int. Cl. G01N 33/569; G01N 15/14; A61L 27/38

(52) U.S. Cl. CPC G01N 33/56966; G01N 15/14; A61L 27/3817; A61L 2430/24; A61L 2300/64; A61L 2430/06; G01N 2015/1486

(57) ABSTRACT

Compositions and methods are provided for preparing and quantifying multipotential stromal cells derived from bone marrow aspirate for use in regenerative therapy and other medical treatments. A rapid, simple assay can be performed intra-operatively to quantify multipotential stromal cells in a sample for determining a dosage of multipotential stromal cells to be administered in regenerative therapeutic compositions.

### CLAIM OF PRIORITY

This application claims the benefit of U.S. Provisional Patent Application Ser. No. 62/502,902, filed on May 8, 2017, the benefit of priority of which is claimed hereby, and which is incorporated by reference herein in its entirety.

### FIELD

This patent document generally pertains to medicine and more particularly, but not by way of limitation, to compositions and methods for improving treatment of various injuries, defects and diseases using multipotential stromal cells, including injuries, defects and diseases in bone, cartilage and joint tissues.

### BACKGROUND

This section provides background information related to the present disclosure, but such background information is not admitted as being prior art.

Although joint replacement is the gold standard treatment for advanced osteoarthritis (OA), other therapeutic methods focus on joint preservation and cartilage repair in OA subjects. Current and emergent regenerative strategies include targeting the affected joint environment with biological modifiers, such as differentiated or undifferentiated progenitor or stem cells and growth factors. Cellular regenerative therapies for OA include the use of chondrocytes or multipotential mesenchymal stromal cells (also known as marrow stromal cells, mesenchymal stem cells, mesenchymal stromal cells, or multipotential mesenchymal stem cells). Chondrocyte-based therapy is popular; however, it has some limitations and requires multiple operations and in vitro manipulation. Multipotential mesenchymal stromal cells have emerged as an alternative to chondrocyte regenerative therapy because multipotential stromal cells are derived from tissue that is abundant and these cells have a higher proliferative capability. Multipotential mesenchymal stromal cells can be derived from bone marrow, bone marrow aspirate, blood and blood fractions (e.g., blood-based autologous protein solutions), bone marrow fractions e.g., bone marrow-based autologous protein solution (multipotential mesenchymal stromal cells derived from these sources are collectively referred to herein as “BM-MSCs”), and adipose tissue, lipoaspirate, and fractions thereof, e.g., blood/saline fraction and Stromal Vascular Fraction (multipotential mesenchymal stromal cells derived from these sources are collectively referred to herein as “AT-MSCs”). BM-MSCs and AT-MSCs (collectively referred to herein as “MSCs”) are most often used for clinical trials in OA, and evidence suggests that BM-MSCs have superior cartilage and bone healing capacity compared to AT-MSCs. Additionally, the rationale for using BM-MSCs in clinical settings of OA relates to the effective results obtained from the microfracture technique that releases endogenous BM-MSCs and growth factors, thereby facilitating cartilage repair and bone repair in patients.

Bone marrow multipotential mesenchymal stromal cells have been shown to be effective for bone repair in animal models of femoral head avascular necrosis (AVN), a pre-OA condition. Clinical application of uncultured BM-MSCs in the form of bone marrow concentrates has been reported to be safe when delivered with or without scaffolds, e.g., collagen, hydrogel, hyaluronic and other synthetic and natural scaffolds, at the site of femoral head AVN. Additionally, several independent clinical studies have shown that BM-MSC therapy improves pain associated with femoral head AVN and decreases the lesion volume of femoral head AVN. Bone marrow aspirates and bone marrow concentrates have demonstrated substantial effectiveness in the healing of other pre-OA conditions, such as osteochondral defects and/or metaphyseal bone defects, in animal models and in human clinical trials. Furthermore, the direct intra-articular injection of bone marrow samples and application of collagen and hyaluronic scaffolds loaded with bone marrow samples have been described as simple, inexpensive and effective procedures to treat both knee and hip OA.

The concentration of bone marrow aspirate has become a popular procedure to increase BM-MSC numbers within a minimum volume of bone marrow, particularly when bone marrow is to be loaded on scaffolds, e.g. collagen or hyaluronic scaffolds. However, the numbers of BM-MSCs in bone marrow aspirates and bone marrow concentrates are widely variable, depending on the site of harvesting, the aspirate volume, aspiration technique, method of concentration, and donor-related factors, e.g., age and gender. Therefore, determining the number of native MSCs in blood, blood fractions, bone marrow, bone marrow fractions, bone marrow aspirate, blood and/or bone marrow concentrates, adipose tissue, lipoaspirate blood/saline fraction and Stromal Vascular Fraction, and other tissues, tissue extracts or tissue fractions that comprise multipotential mesenchymal stromal cells, is a means to optimise multipotential mesenchymal stromal cell therapy and improve clinical outcomes with minimum cost and time.

Despite all the advantages of bone marrow aspirates, bone marrow concentrates, blood fractions and bone marrow fractions, adipose tissue, lipoaspirate and adipose tissue fractions (e.g., blood/saline fraction and Stromal Vascular Fraction), the optimal therapeutic dose of MSCs derived from these tissues, whether delivered directly or loaded on scaffolds, remains generally unknown and is poorly controlled. Although the colony forming unit-fibroblast (CFU-F) assay is currently considered the gold standard for enumeration of MSCs, the CFU-F assay requires fourteen days of culturing the bone marrow sample before reliable information can be ascertained regarding the number of MSCs in the bone marrow sample. Thus, there is a need for a method to accurately and quickly quantify the number of MSCs in a tissue sample. Furthermore, there is a need for a method to accurately enumerate the number of MSCs in a tissue sample that can be performed rapidly and proximate to the time of therapeutic application, e.g., intra-operatively or within less than 14 days of anticipated therapeutic use. There is also a need for compositions for rapid preparation of tissue samples for enumeration of MSCs in the tissue sample.

### OVERVIEW

This overview is intended to provide an overview of subject matter of the present patent application. It is not intended to provide an exclusive or exhaustive explanation of the present technology, the invention or its full scope or all its features. The detailed description is included to provide further information about the present technology.

The compositions and methods of the present technology provide an assay that permits rapid, accurate, and automated counting of multipotential mesenchymal stromal cells in a tissue sample that comprises MSCs. The method can include staining a tissue sample, e.g., bone marrow sample or adipose tissue sample, using a fluorescence activated cell sorting panel comprising fluorescent antibodies to CD45 and CD271, and a nucleated cell dye, e.g., Vybrant® DyeCycle™ Ruby, with staining for 5 minutes, after which MSCs can be enumerated by immediately counting the stained tissue sample using automated flow cytometry with gating for CD45^{-/low/dim} and CD271^{+/high/bright} and acquiring events in 10 minutes or less to provide a 15 minute assay. Automated cell counts can be verified using a standard colony forming unit-fibroblast assay (CFU-F). Using the fast enumeration assay described herein, the MSC percentage from bone marrow aspirate samples was found to be 0.001-0.07%, with a median of 0.016%. The number of MSCs obtained from bone marrow aspirate samples using automated flow cytometry (median=1,696 MSCs/ml, range=64-20,992 MSCs/ml) significantly correlates with the number of MSCs counted by a standard CFU-F assay (r=0.7237, p=0.0004). The enumeration of MSCs before and after concentration of bone marrow samples using a BioCUE® concentrator (Zimmer Biomet) evidences a mean 5-fold increase in MSCs. Additionally, the fast enumeration assay described herein can detect variable levels of MSC attachment to a scaffold (e.g., Bio-Gide®, Zimmer Biomet) that correlates with the number of cells that colonize the scaffold (p=0.0348, r=0.8434). The simple, fast enumeration assay described herein can be used for accurate intra-operative enumeration of MSCs in bone marrow samples, bone marrow concentrates, bone marrow fractions (e.g., bone-marrow autologous protein solutions), blood, blood fractions, and blood-derived autologous protein solutions), adipose tissue, lipoaspirate fractions (e.g., blood/saline fraction and Stromal Vascular Fraction), and any other tissue that comprises MSCs that are positive for, or evidence high expression of, CD271 CD29, CD44, CD40a-f, CD51, CD73, CD 90, CD105, CD106, CD146, CD166, CD200, Strol, or any other biomarker that is positively expressed (e.g., highly expressed) in MSCs (referred to herein as a “positive marker” of MSCs), and that is negative for, or evidences low expression of, at least one of CD45, CD11b, CD14, CD19, CD31, CD33, CD34, CD79α, and HLA-DR, or any other cellular biomarker that is not expressed (or is of low expression) in MSCs (referred to herein as a “negative marker” of MSCs).

The fast enumeration assay described herein can be used to standardize dosing of MSCs and improve the outcomes of any and all MSC therapies including, without limitation, those for wound healing, cartilage or bone repair, spinal disorders, spinal injury and brain injury, and degenerative disc disease, neurodegenerative diseases (e.g., Parkinson's, Amyotrophic lateral sclerosis, and Alzheimer's), heart disease, and atherosclerotic or peripheral vascular disease and critical limb ischemia, graft vs. host disease, autoimmune diseases, and Crohn's disease. The fast enumeration assay can also be used to standardize dosing of hematopoietic stem cells for use in hematopoietic stem cell therapies. For example, different types of joint degeneration including OA, AVN and osteochondral defects can be treated using minimally-manipulated tissue samples (e.g., bone marrow, adipose tissue, or the fractions, aspirates or concentrates of bone marrow or adipose tissue), with or without incorporation of the tissues (or cells derived from the tissues) in/on scaffolds. However, the quantity of implanted MSCs that will produce the best outcomes in these conditions has yet to be determined. The fast enumeration assay described herein for enumerating MSCs in tissue samples is reliable and ideal for the clinical applications to address this problem and to standardize dosages of MSCs in clinical application. This fast enumeration assay will help significantly improve regenerative therapies, including those involving vascular, musculoskeletal, cartilage and bone repair and regeneration.

To further illustrate the methods, compositions and systems disclosed herein, a non-limiting list of aspects of the invention provided here:

Aspect 1 can include or use a method for quantifying MSCs in a tissue sample comprising contacting a tissue sample with a first detecting reagent configured to detect to a positive cellular marker that is highly expressed in MSC, and a second detecting reagent configured to detect a negative cellular marker that is absent or has low expression in MSCs, and counting cells in the tissue sample using flow cytometry, wherein the contacting and counting are completed in 20 minutes or less.

Aspect 2 can include or use, or can optionally be combined with the subject matter of Aspect 1 to optionally include or use, a method wherein red blood cells in the tissue sample are not removed or lysed before counting the cells.

Aspect 3 can include or use, or can optionally be combined with the subject matter of any one or a combination of Aspects 1 or 2 to optionally include or use, a method wherein the positive cellular marker comprises CD271, CD29, CD44, CD40a-f, CD51, CD73, CD90, CD105, CD106, CD146, CD166, CD200, STRO1, or a combination thereof.

Aspect 4 can include or use, or can optionally be combined with the subject matter of any one or a combination of Aspects 1 through 3 to optionally include or use, a method wherein the negative cellular marker comprises CD45, CD11b, CD14, CD19, CD31, CD33, CD34, CD79α, HLA-DR, or a combination thereof.

Aspect 5 can include or use, or can optionally be combined with the subject matter of any one or a combination of Aspects 1 through 4 to optionally include or use, a method wherein the contacting further includes contacting the tissue sample with a third detecting reagent configured to only detect DNA or cells having a nucleus.

Aspect 6 can include or use, or can optionally be combined with the subject matter of any one or a combination of Aspects 1 through 5 to optionally include or use, a method wherein the negative cellular marker is highly expressed in another cell type hat is present in the tissue sample.

Aspect 7 can include or use, or can optionally be combined with the subject matter of any one or a combination of Aspects 1 through 6 to optionally include or use, a method wherein the contacting and counting are completed within about 15 minutes or less.

Aspect 8 can include or use, or can optionally be combined with the subject matter of any one or a combination of Aspects 1 through 7 to optionally include or use, a method wherein the flow cytometer is configured such that the specified number of acquisition events are obtained in about 10 minutes or less.

Aspect 9 can include or use, or can optionally be combined with the subject matter of any one or a combination of Aspects 1 through 8 to optionally include or use, a method further comprising administering the tissue sample to a subj ect.

Aspect 10 can include or use, or can optionally be combined with the subject matter of any one or a combination of Aspects 1 through 9 to optionally include or use, a kit for quantifying multipotential stromal cells (MSCs) comprising a first reagent configured to detect a positive cellular marker that is highly expressed in MSCs, a second reagent configured to detect a negative cellular marker that is absent or weakly expressed in MSCs but that is highly expressed in another cell type in blood, bone marrow, or adipose tissue, and a third reagent configured to detect nucleated cells, wherein the first reagent, second reagent, and third reagent are packaged together.

Aspect 11 can include or use, or can optionally be combined with the subject matter of any one or a combination of Aspects 1 through 10 to optionally include or use, a kit wherein the positive cellular marker comprises CD271, CD29, CD44, CD40a-f, CD51, CD73, CD90, CD105, CD106, CD146, CD166, CD200, STRO1, or a combination thereof.

Aspect 12 can include or use, or can optionally be combined with the subject matter of any one or a combination of Aspects 1 through 11, to optionally include or use, a kit wherein the negative cellular marker comprises

CD45, CD11b, CD14, CD19, CD31, CD33, CD34, CD79α, HLA-DR, or a combination thereof.

Aspect 13 can include or use, or can optionally be combined with the subject matter of any one or a combination of Aspects 1 through 12 to optionally include or use, a kit further comprising counting beads.

Aspect 14 can include or use, or can optionally be combined with the subject matter of any one or a combination of Aspects 1 through 13 to optionally include or use, a kit wherein the first reagent, the second reagent, and the third reagent are provided in dried form.

Aspect 15 can include or use, or can optionally be combined with the subject matter of any one or a combination of Aspects 1 through 14 to optionally include or use, a kit wherein the first reagent, the second reagent and the third reagent are premixed.

Aspect 16 can include or use, or can optionally be combined with the subject matter of any one or a combination of Aspects 1 through 15 to optionally include or use, a kit wherein the package comprises a tube configured for use in a flow cytometer.

Aspect 17 can include or use, or can optionally be combined with the subject matter of any one or a combination of Aspects 1 through 16 to optionally include or use, a kit wherein the first reagent, the second reagent and the third reagent are each provided in amount sufficient to saturate a 100 μl tissue sample.

Aspect 18 can include or use, or can optionally be combined with the subject matter of any one or a combination of Aspects 1 through 17 to optionally include or use, a method for regenerative therapy using multipotential stromal cells (MSCs), the method comprising: contacting a portion of a tissue sample with a first reagent configured to identify a first marker that is highly expressed in MSCs, a second reagent configured to identify a second marker that is absent or weakly expressed in MSC but is highly expressed in another cell type in the tissue sample, and a third reagent configured to identify only nucleated cells in the tissue sample; quantifying the number of MSCs in the tissue sample or the concentration of MSCs in the tissue sample using flow cytometry; and administering the tissue sample to a subj ect.

Aspect 19 can include or use, or can optionally be combined with the subject matter of any one or a combination of Aspects 1 through 18 to optionally include or use, a method wherein the quantifying is performed proximal to the time of administering the tissue sample to the subject.

Aspect 20 can include or use, or can optionally be combined with the subject matter of any one or a combination of Aspects 1 through 19 to optionally include or use, a method wherein the tissue sample is incubated with a scaffold, and the method further comprises determining the number of MSCs adsorbed on the scaffold.

Aspect 21 can include or use, or can optionally be combined with any portion or combination of any portions of any one or more of Aspectsl through 20 to include or use, subject matter that can include means for performing any one or more of the functions of Aspects 1 through 24, or a machine-readable medium including instructions that, when performed by a machine, cause the machine to perform any one or more of the functions of Aspects 1 through 24.

Further areas of applicability will become apparent from the description provided herein. The description and specific examples in this summary are intended for purposes of illustration only and are not intended to limit the scope of the present disclosure.

### DRAWINGS

The drawings described herein are for illustrative purposes only of selected examples and not all possible examples or implementations, and these drawings are not intended to limit the scope of the present disclosure.

FIG. 1A is a graph showing the numbers of MSCs counted in an example of the fast enumeration assay using anti-CD45 antibody staining with various dye volumes (one-way ANOVA test; sample number=10). Cells counts performed by Attune® Flow Cytometer (Invitrogen/Thermo Fischer Scientific, Inc.).

FIG. 1B is a graph showing the numbers of MSCs counted in an example of the fast enumeration assay using anti-CD271 antibody staining with various dye volumes (one-way ANOVA test; sample number=8). Cells counts performed by Attune® Flow Cytometer.

FIG. 1C is a graph showing the numbers of MSCs counted in an example of the fast enumeration assay using 2.5 mM Vybrant® DyeCycle™ Ruby (VD Ruby) staining with various dye volumes (one-way ANOVA test; sample number=8). Cells counts performed by Attune® Flow Cytometer.

FIG. 1D is a graph showing the number of MSCs counted after one-step staining at room temperature, and after two-step staining at room temperature and then at 37° C. (paired t test, sample number=6) in examples of a one-step fast enumeration assay and a two-step fast enumeration assay. Cell counts performed by Attune® Flow Cytometer.

FIG. 1E is a graph showing the number of MSCs counted relative to staining at different temperatures (one-way ANOVA test; sample number=7). Cell counts performed by Attune® Flow Cytometer.

FIG. 1F is a graph showing the number of MSCs counted relative to staining times of 5, 10 and 15 minutes (one-way ANOVA test; sample number=10). Cell counts performed by Attune® Flow Cytometer.

FIG. 1G is a graph showing the number of MSCs counted relative to counting beads (control) and automated counting using an example of the fast enumeration assay (Wilcoxon matched-pairs signed rank test; sample number=9). Cell counts performed by Attune® Flow Cytometer.

FIG. 1H is a graph showing the number of MSCs counted in bone marrow aspirate samples that are undiluted, diluted 5-fold, and diluted 10-fold (sample number=5). Cell counts performed by Attune®Flow Cytometer.

FIG. 2A shows scatter plots of MSC counts from a high quantity of MSC samples detected using automated counting according to an example of the fast enumeration assay, and a CFU assay plate. Cell counts performed by Attune® Flow Cytometer, LSRII Flow Cytometer (BD Biosciences) and by CFU-F assay.

FIG. 2B shows scatter plots of MSC counts from a low quantity of MSC samples detected using automated counting according to an example of the fast enumeration assay, and by CFU assay plate. Cell counts performed by Attune® Flow Cytometer, LSRII Flow Cytometer (BD Biosciences) and by CFU-F assay.

FIG. 2C is a dot-plot showing Spearman rank correlation analysis for MSC numbers quantified by Attune® Flow Cytometer in a fast enumeration assay, compared with MSC numbers quantified using a 40 minute assay with cells counted on an LSRII Flow Cytometer (sample number=33).

FIG. 2D is a dot-plot showing Spearman rank correlation analysis for MSC number quantified by Attune® Flow Cytometer compared with a CFU-F assay (sample number=33).

FIG. 3A shows dot-plots of the correlation of female donors by age when MSCs are counted by Attune® Flow Cytometer (sample number=18) and LSRII Flow Cytometer (sample number=16), using an example of the fast enumeration assay, and MSCs counted by a standard CFU-F assay (sample number=14).

FIG. 3B shows dot-plots of the correlation of male donor by age when MSC are counted by Attune® Flow Cytometer (sample number=19) and LSRII Flow Cytometer (sample number=14) using an example of the fast enumeration assay, and MSCs counted by a standard CFU-F assay (sample number=12).

FIG. 4A is a bar graph showing MSC counts performed using an example of the fast enumeration assay of bone marrow concentrates prepared using a concentrator (BioCUE®, Zimmer Biomet) that were undiluted, 5-fold diluted, or 10-fold diluted (sample number=9).

FIG. 4B is a graph showing the numbers of MSCs counted using an example of the fast enumeration assay of bone marrow samples, pre-concentration and post-concentration, using a concentrator (BioCUE ®, ZimmerBiomet) (Wilcoxon matched-pairs signed-rank test; sample number=15).

FIG. 4C is a graph showing the fold-increase of MSC numbers after concentration of bone marrow samples using a concentrator (BioCUE ®, ZimmerBiomet) comparing MSC numbers counted using an example of the fast enumeration assay (automated counting), and CFU-F assay (paired t test analysis; sample number=11 samples).

FIG. 4D is a dot-plot showing the mean fold-increase of MSCs counted using an example of the fast enumeration assay (automated counting), and platelets counted using Sysmex (Sysmex, Inc.) following concentration (BioCUE®, ZimmerBiomet) (sample number=15 for MSCs; sample number=9 for platelets).

FIG. 5A shows scatter plots of MSC numbers counted using an example of the fast enumeration assay with gating for CD45^{-/low/dim} and CD271^{+/high/bright} before (pre-loading) and after (post-loading) loading of a scaffold with bone marrow sample.

FIG. 5B is a bar graph showing the number of MSC per ml of bone marrow sample pre-loading and attached to a scaffold using an example of the fast enumeration assay with gating for CD45^{-/low/dim} and CD271^{+high/bright}.

FIG. 5C is a dot-plot showing the number of MSCs attached to a scaffold relative to the number of surviving MSCs on the scaffold after 2 weeks in culture using an example of the fast enumeration assay with gating for CD45^{-/low/dim} and CD271^{+/high/bright} (Pearson r test; sample number=6).

FIG. 6 is an example of a flow cytometry kit as described herein including one or more fluorescent antibodies to a positive marker of MSC, one or more fluorescent antibodies to a negative marker of MSC, and optionally a nucleated cell stain, a live cell or dead cell stain, a cell phase stain, counting beads, or a combination thereof.

### DETAILED DESCRIPTION

The present technology provides validated, simple and rapid flow cytometry assays to quantify multipotential stromal (mesenchymal stem) cells that can be used in clinical settings and performed proximal to (e.g., within the limited intra-operative timeframe of 48 hours or less) therapeutic use of multipotential stromal cells derived from blood and blood fractions, bone marrow, bone marrow aspirates and fractions thereof, blood and/or plasma and/or bone marrow concentrates, adipose tissue, lipoaspirate and/or fractions thereof (e.g., blood/saline fraction and Stromal Vascular Fraction of lipoaspirate), and lipoaspirate or lipoaspirate fraction concentrates. Multipotential mesenchymal stromal cells derived from any source tissue, including, without limitation, adipose tissue, blood, bone marrow, and fractions or concentrates of blood and/or bone marrow and/or adipose tissue, are collectively referred to herein as “multipotential mesenchymal stromal cells” or “MSCs.”

Low affinity nerve growth factor receptor (CD271) is considered one of most specific markers for MSCs, and CD271 has been used to select MSCs with high proliferative capacity. CD271 can be used according to the fast enumeration assay methods described herein to distinguish between MSCs and hematopoietic stem cells isolated from blood, bone marrow, adipose and other tissue samples comprising MSCs. The protein sequence for human CD271 (also known as tumor necrosis factor receptor superfamily member 16) is well known, and can be generally described by the following protein sequence:

MGAGATGRAMDGPRLLLLLLLGVSLGGAKEACPTGLYTHSGECCKACNLG
EGVAQPCGANQTVCEPCLDSVTFSDVVSATEPCKPCTECVGLQSMSAPCV
EADDAVCRCAYGYYQDETTGRCEACRVCEAGSGLVFSCQDKQNTVCEECP
DGTYSDEANHVDPCLPCTVCEDTERQLRECTRWADAECEEIPGRWITRST
PPEGSDSTAPSTQEPEAPPEQDLIASTVAGVVTTVMGSSQPVVTRGTTDN
LIPVYCSILAAVVVGLVAYIAFKRWNSCKQNKQGANSRPVNQTPPPEGEK
LHSDSGISVDSQSLHDQQPHTQTASGQALKGDGGLYSSLPPAKREEVEKL
LNGSAGDTWRHLAGELGYQPEHIDSFTHEACPVRALLASWATQDSATLDA
LLAALRRIQRADLVESLCSESTATSPV.

CD45 (protein tyrosine phosphatase, receptor type C) is a hematopoietic cell marker that can be used according to the fast enumeration assay methods described herein to distinguish between MSCs and hematopoietic stem cells isolated from blood, bone marrow, adipose and other tissue samples comprising MSCs and hematopoietic stem cells. The protein sequence for human isoforms of CD45 are well known, for example human CD45 (isoform 1) has the following protein sequence:

MTMYLWLKLL AFGFAFLDTE VFVTGQSPTP SPTGLTTAKM
PSVPLSSDPL PTHTTAFSPASTFERENDFS ETTTSLSPDN
TSTQVSPDSL DNASAFNTTG VSSVQTPHLP
THADSQTPSAGTDTQTFSGS AANAKLNPTP GSNAISDVPG
ERSTASTFPT DPVSPLTTTL SLAHHSSAALPARTSNTTIT
ANTSDAYLNA SETTTLSPSG SAVISTTTIA TTPSKPTCDE
KYANITVDYLYNKETKLFTA KLNVNENVEC GNNTCTNNEV
HNLTECKNAS VSISHNSCTA PDKTLILDVPPGVEKFQLHD
CTQVEKADTT ICLKWKNIET FTCDTQNITY REQCGNMIED
NKEIKLENLE PEHEYKCDSE ILYNNHKFTN ASKIIKTDFG
SPGEPQIIFC RSEAAHQGVI TWNPPQRSFEINFTLCYIKET
EKDCLNLDKN LIKYDLQNLK PYTKYVLSLH AYIIAKVQRN
GSAAMCHFTTKSAPPSQVWN MTVSMTSDNS MHVKCRPPRD
RNGPHERYHL EVEAGNTLVR NESHKNCDFRVKDLQYSTDY
TFKAYFHNGD YPGEPFILHH STSYNSKALI AFLAFLIIVT
SIALLVVLYKIYDLHKKRSC NLDEQQELVE RDDEKQLMNV
EPIHADILLE TYKRKIADEG RLFLAEFQSIPRVFSKFPIK
EARKPFNQNK NRYVDILPYD YNRVELSEIN GDAGSNYINA
SYIDGFKEPRKYIAAQGPRD ETVDDFWRMI WEQKATVIVM
VTRCEEGNRN KCAEYWPSME EGTRAFGDVVVKINQHKRCP
DYIIQKLNIV NKKEKATGRE VTHIQFTSWP DHGVPEDPHL
LLKLRRRVNAFSNFFSGPIV VHCSAGVGRT GTYIGIDAML
EGLEAENKVD VYGYVVKLRR QRCLMVQVEAQYILIHQALV
EYNQFGETEV NLSELHPYLH NMKKRDPPSE PSPLEAEFQR
LPSYRSWRTQ HIGNQEENKS KNRNSNVIPY DYNRVPLKHE
LEMSKESEHD SDESSDDDSD SEEPSKYINA SFIMSYWKPE
VMIAAQGPLK ETIGDFWQMI FQRKVKVIVM LTELKHGDQE
ICAQYWGEGKQTYGDIEVDL KDTDKSSTYT LRVFELRHSK
RKDSRTVYQY QYTNWSVEQL
PAEPKELISMIQVVKQKLPQKNSSEGNKHH KSTPLLIHCR
DGSQQTGIFC ALLNLLESAE TEEVVDIFQVVKALRKARPG
MVSTFEQYQF LYDVIASTYP AQNGQVKKNN HQEDKIEFDN
EVDKVKQDANCVNPLGAPEK LPEAKEQAEG SEPTSGTEGP
EHSVNGPASP ALNQGS.

MSCs express high levels of CD271 (denoted in flow cytometry as CD271^{+/high/bright}), while hematopoietic progenitor cells express low levels of CD271 (denoted in flow cytometry as CD271^{-/low/dim}) Conversely, hematopoietic progenitor cells express high levels of CD45 (denoted in flow cytometry as CD45^{+/high/bright})and MSCs express low levels of CD45 (denoted in flow cytometry as CD45^{-/low/dim}). However, because CD271 is also expressed by other cells in some tissue samples, e.g., bone marrow and adipose tissue, automated flow cytometry selection and/or counting MSCs in bone marrow or adipose tissue samples using only CD271 can result in detection of CD271-positivve cells types other than MSCs. Thus, the present technology uses a combination of CD271 and CD45, or one or more other biomarkers that are highly expressed in another cell type of bone marrow or adipose tissue, but that are not highly expressed in MSCs (i.e., a negative biomarker of MSCs) to discriminate between MSCs and other cell types in a tissue sample. Thus, a combination of flow cytometry reagents (e.g., fluorescent stains) can be used to detect a combination CD271 and a negative biomarker of MSC (e.g., CD45) to distinguish MSCs from one or more other cell types in bone marrow tissue samples and/or adipose tissue samples (as used herein a tissue sample includes a tissue aspirate, a fraction or fluid derived from the tissue, or a concentrate of a tissue or a tissue fraction). Similarly, fluorescent staining for a combination of CD271 and CD45 can allow for selective sorting and/or counting of hematopoietic stem cells (CD45^{+/high/bright}) for enumeration of hematopoietic stems cell for use in hematopoietic stem cell therapies.

Based on the selective fluorescent flow cytometry gating for the combination of positive or high or bright expression of a biomarker of MSC, e.g., CD271 (CD271^{+/high/bright}), and/or the negative or low or dim expression of a second biomarker that is a negative biomarker for MSC (e.g., CD45^{-/low/dim}), the second biomarker being positively (moderately or highly) expressed in a cell type other than a MSC (e.g., a hematopoietic stem cell), the present inventors have developed methods to quickly prepare tissue samples for automated MSC quantification and to rapidly and accurately quantify the numbers of MSCs in tissue samples. Additionally, the methods and assays of the present invention can be used to calculate the number of MSCs in tissue concentrates, e.g., bone marrow or adipose tissue concentrates, and the numbers of MSCs attached to a scaffold (e.g., a hyaluronic scaffold or collagen scaffold, such as Bio-Gide®, after loading the scaffold with a tissue sample comprising MSCs. The assays and methods can be used in clinical applications and can be performed using readily available, compact flow cytometers, allowing for automated and rapid counting of MSCs for various MSC therapies, including, without limitation, bone and cartilage regenerative therapies, bone grafting with autograft or allograft tissues, and for use as an autologous anti-inflammatory (AAI) therapeutic agent.

Methods for Harvesting Tissue Samples

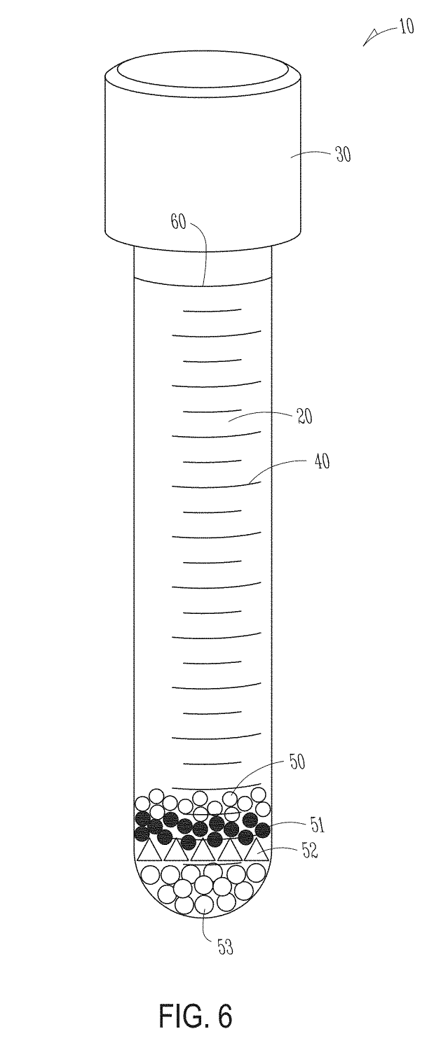

In one example, a method of preparing a tissue sample for enumeration of MSCs can include harvesting a volume of bone marrow aspirate (e.g., 1-25 ml) from a donor, e.g. from the posterior iliac crest of a donor, and distributing the sample into one or more EDTA blood collection tubes as described in Cuthbert, R. J., et al., Examining the feasibility of clinical grade CD271+ enrichment of mesenchymal stromal cells for bone regeneration. PLoS One, 2015; 10(3): p. e0117855. In another example of a method for enumerating MSCs, for bone marrow samples to be used for preparing bone marrow concentrate, more than 25 ml (e.g., 30-100 ml or more) of bone marrow can be harvested from the posterior iliac crest of a donor and distributed into one or more EDTA blood collection tubes. In another example, a bone marrow sample can be passed through a cell strainer (60) (see FIG. 6) e.g., 70 μm cell strainer (BD Biosciences) to ensure that the bone marrow sample does not contain clots.

In another example, a volume of adipose tissue or lipoaspirate can be harvested from a donor. For example, adipose tissue can be collected by suction-assisted tumescent liposuction inside a specialized collection container attached to suction hoses and to a liposuction cannula. A collection container (10) can have a filter (60) that allows the tumescent fluid (blood/saline fraction) to pass through and to retain the solid adipose tissue. In another example, a filter can have a suitable pore size for retaining adipose tissue, e.g., approximately a 100 μm pore size. In one example, after collecting the adipose tissue, a collection container can be removed from a suction device and can be reattached to a centrifugation device, e.g., a tube (20), or the adipose tissue can be removed from the collection tube and deposited in a container suitable for centrifugation. In one example, the collected adipose tissue can be enzymatically treated or sonicated and then centrifuged (e.g., at 300 g×5 minutes), and a pellet containing cells (the Stromal Vascular Fraction) can then be resuspended in a biocompatible solution (e.g., saline, growth medium, stem cell expansion medium, blood, plasma, plasma concentrate, platelet-rich plasma, bone marrow aspirate, or bone marrow concentrate), and the resuspended cells can be quantified in an example of a fast enumeration assay as described herein.

Various methods and devices that can be used in the present technology for isolating and/or fractionating adipose tissue include those as described by U.S. Pat. No. 7,374,678, Leach, issued May 20, 2008; U.S. Pat. No. 7,179,391 to Leach et al., issued Feb. 20, 2007; U.S. Pat. No. 7,992,725, Leach et al., issued Aug. 9, 2011; U.S. Pat. No. 7,806,276, Leach et al., issued Oct. 5, 2010; and U.S. Pat. No. 8,048,297, Leach et al., issued Nov. 1, 2011. A device, such as the GPS™ Platelet Concentrate System, commercially available from Biomet Biologics, LLC (Warsaw, Ind., USA), can also be used to concentrate lipoaspirate.

In another example, MSCs from a blood/saline fraction of lipoaspirate can be isolated as described in Francis, M. et al., Isolating adipose-derived mesenchymal stem cells from lipoaspirate blood and saline fraction, Organogensis (2010); 6(1):11-14. In one example, a cell pellet obtained from the blood/saline fraction of lipoaspirate can be resuspended in a lysis solution (e.g., NH₄CL) for 5 minutes to allow lysis of red blood cells, and the solution can be centrifuged (400 g×10 minutes) to obtain a pellet of cells that can be resuspended in a biocompatible solution. In another preferred example, a blood/saline fraction collected from lipoaspirate can be centrifuged to form a cell pellet (e.g., at 400 g×10 minutes), and the cell pellet resuspended in a biocompatible solution (e.g., saline, growth medium, blood, plasma, plasma concentrate, or platelet-rich plasma) without lysis or removal of RBCs from the blood/saline fraction, and the resuspended cell pellet can then be processed in an example of a fast enumeration assay as described herein.

Using Flow Cytometry for a Fast Enumeration Assay for MSCs

In one aspect, a volume of a tissue sample, e.g., whole bone marrow or lipoaspirate fraction, without red blood cells (RBCs), can be used for enumerating MSCs. In one example, RBCs can be removed from a tissue sample by lysis, filtration, sedimentation, centrifugation, or other known methods to remove RBCs from the tissue sample, e.g., bone marrow or whole blood or lipoaspirate. In another example, a tissue sample can be processed such that RBC lysis occurs, e.g., using a lysis composition selective for lysis of RBCs. In another example, a tissue sample that has been treated with a lysis solution can be washed after lysis treatment and before further processing, e.g., processing with fluorescent antibody staining. In other examples, a tissue sample can be processed with fluorescent antibody staining without first washing the tissue sample after treatment with a lysis solution. In a preferred example, a tissue sample containing intact RBCs or RBCs that have not been lysed can be processed with fluorescent antibody staining or other stains or dyes without first removing or lysing RBCs. In still more preferred examples, a tissue sample can be processed with fluorescent antibody staining, and/or other stains or dyes without removing RBCs or without treating the RBCs with a lysis buffer. In still other preferred examples, a tissue sample includes intact RBCs at the time the tissue sample is processed through a flow cytometer.

In one example, an aliquot of a tissue sample (e.g., 10 μl to 500 μl, preferably 100 μl) can be processed with one or more detecting reagents configured to detect a biomarker in one or more cells of a tissue sample using flow cytometry, e.g. fluorescence-activated cell sorting (FACS). Fluorophore-conjugated antibodies (e.g., directed against various cellular biomarkers) and other dyes and stains configured to detect various cellular components (e.g., DNA, RNA, proteins) are commercially available from many sources, e.g., ThermoFisher Scientific, BD Biosciences, Bio-Rad, Beckman Coulter, R&D Systems, and Miltenyi Biotec, and other sources (sometimes individually or collectively fluorophore-conjugated reagents, and other dyes and stains are referred to herein as “detecting reagents”).

In one aspect, a tissue sample can be stained with a detecting reagent directed against or configured to detect one or more known positive biomarkers of MSC phenotype i.e., a biomarker that is highly expressed in MSCs (a biomarker in MSCs that stains +/high/bright). In an example, a positive biomarker of MSCs can comprise CD271, CD29, CD44, CD40a-f, CD51, CD73, CD90, CD105, CD106, CD146, CD166, CD200, STRO1, or a combination thereof. In a preferred example, a detecting reagent can comprise an antibody directed against CD271, CD29, CD44, CD40a-f, CD51, CD73, CD90, CD105, CD106, CD146, CD166, CD200, STRO1, or a combination thereof. In a preferred example, a positive biomarker of MSC can comprise CD271, and a detecting reagent in a fast enumeration assay can comprise a fluorophore-conjugated anti-CD271 antibody. In another example, a tissue sample can be stained with one or more detecting reagents directed against or otherwise configured to detect one or more known negative biomarkers of MSCs (a biomarker in MSCs that stains −/low/dim). In a preferred example, a negative biomarker of MSCs comprises a biomarker that is highly expressed on another cell type (+/high/bright) in the tissue sample, i.e., a cell type that is not an MSC. In one example, a negative biomarker of MSC can comprise CD45, CD11b, CD14, CD19, CD31, CD33, CD34, CD79α, HLA-DR, or a combination thereof. In another example, a detecting reagent configured to detect a negative biomarker of MSC can comprise anti-CD45 antibody, anti-CD11b antibody, anti-CD14 antibody, anti-CD19 antibody, anti-CD31 antibody, anti-CD33 antibody, anti-CD34 antibody, anti-CD79a antibody, anti-HLA-DR antibody, or a combination thereof. In a preferred example, a negative biomarker of MSC can comprise CD45 and a detecting reagent configured to detect a negative biomarker of MSCs can comprise anti-CD45 antibody. In a preferred example, an aliquot of a tissue sample can be processed with one or more detecting reagents configured to detect a combination of a positive marker for MSCs (+/high/bright) and a negative biomarker of MSCs (−/low/dim). In a preferred example, an aliquot of a tissue sample can be processed with one or more detecting reagents configured to detect CD271 (e.g., a fluorescent conjugated anti-CD271 antibody) and CD45 (e.g., fluorescent conjugated anti-CD45 antibody).

In another aspect, an aliquot of a tissue sample can be processed with one or more detecting reagents configured to only detect nucleated cells. In another example, an aliquot of a tissue sample can be processed with one or more detecting reagents configured to gate out (not detect) RBCs and/or platelets. In another example, a detecting reagent can be configured to detect DNA (e.g., a DNA-selective stain or dye) or can be configured to only detect nucleated cells, thereby gating out RBCs and platelets during flow cytometry, such that it is not necessary to remove RBCs or lyse RBCs before or after staining of other cells. In an example, an aliquot of a tissue sample can be processed with Vybrant® DyeCycle™ Ruby dye, Nuclear Red dye, Nuclear Orange dye, Nuclear Yellow dye, Nuclear green dye, and the like. In a preferred example, a detecting reagent to gate out RBCs and platelets can comprise Vybrant® DyeCycle™ Ruby dye (Thermo Fisher Scientific).

In aspect, staining of a tissue sample with a stain or dye, including a fluorophore-conjugated antibody, can be performed according to a manufacturer's recommended staining conditions (time of staining, temperature of staining and amount or volume of dye or stain used) for a dye or stain. In one example, the temperature of staining can be at a temperature between about 4° C. and about 37° C.

In a preferred example of a fast enumeration assay, the temperature of staining can be at room temperature (e.g., at a temperature between about 15° and about 30° C.). In one example, the time of staining can be for a period of about 5 to 30 minutes. In another example of a fast enumeration assay, staining can be for a period between about 5 minutes and about 15 minutes. In a preferred example, staining can be for a period between about 5 minutes an about 10 minutes. In certain preferred examples of the fast enumeration assay, staining times and staining temperatures can be selected to permit MSCs to be stained and acquired/counted by an automated flow cytometer in a total of 30 minutes or less, preferably in a total of 25 minutes or less, more preferably in a total of 20 minutes or less, and most preferably in a total of 15 minutes or less. In a preferred example, a fast enumeration assay can comprise staining a tissue sample for a period of about 5 minutes and processing the tissue sample in the flow cytometer (acquiring/counting cells) for about 10 minutes. In a preferred example, processing a tissue sample using flow cytometry can consist of staining a tissue sample for 5 minutes with a detecting reagent configured to detect a positive biomarker of MSCs and a negative biomarker of MSCs, and a detecting reagent configured to detect DNA or nucleated cells, and processing the tissue sample through a flow cytometer configured to acquire/count 10,000 to 250,000 events or more in a period of 10 minutes or less, such that the total fast enumeration is completed in about 15 minutes or less.

In another aspect, in an example of a fast enumeration assay flow cytometry can be used to determine the concentration of MSCs in a tissue sample. In one example, commercially available counting beads, e.g., CountBright™ Absolute counting Beads (Thermo Fisher Scientific) of a specified concentration and volume can optionally be added to a tissue sample as a control for automated counting of MSC and to determine MSC concentration in a tissue sample. In one example, 50 μL of counting beads can be added to a tissue sample prior to processing through a flow cytometry device.

An automated flow cytometer can be used for fast, automated cell counting of MSCs processed as described herein, e.g., acoustic focusing flow cytometer Attune® (Invitrogen/Thermo Fisher Scientific, Inc.). Any commercially available automated flow cytometer can be used to enumerate MSCs prepared by the fast enumeration assays and methods described herein. The number of MSCs can be calculated according to the flow cytometer manufacture's equations with consideration of counting bead concentration, sample volume and acquired events for MSCs and counting beads. In a preferred example, a flow cytometer can be configured such that at least about 10,000 to 250,000 events, or more, can be acquired in about 10 minutes or less. Data analysis to determine MSC numbers and concentrations can be performed on standard flow cytometric software, e.g. software provided by the manufacturer of the automated flow cytometer.

Fast Enumeration Assay Validation by LSRII Assay

In certain examples, to validate the efficacy and accuracy of the fast enumeration assay, MSC numbers obtained by the fast enumeration assays and methods disclosed herein can be compared against MSC numbers enumerated by previously published MSC enumeration methods, e.g., as described by Cuthbert, R., et al., Single-platform quality control assay to quantify multipotential stromal cells in bone marrow aspirates prior to bulk manufacture or direct therapeutic use. Cytotherapy (2012) 14(4):431-40 (referred to herein as the “LSRII assay” or “LSRII method”), which is incorporated herein by this reference. In the LSRII assay, fluorescent anti-CD45 antibodies and fluorescent anti-CD271 antibodies are added to bone marrow aspirates for 15 minutes before the lysis of RBCs and before counting beads are added to bone marrow samples. The data acquisition is performed using a flow cytometer, e.g., LSRII (BD Biosciences) and analysed using FACSDiva software (BD Biosciences).

Fast Enumeration Assay Validation by Colony Forming Unit-Fibroblast Assay

In some examples, the colony forming unit-fibroblast (CFU-F) assay can employed and resulting CFU-F numbers compared to MSC numbers obtained using the fast enumeration assays described herein to validate the efficacy and accuracy of the fast enumeration assay. In one example of a CFU-F assay, a volume (e.g., 400 μl) of tissue sample can added to a volume (e.g., 30 ml) of stem cell expansion media (e.g., MACS; Miltenyi-Biotec) then divided into one or more culture dishes, e.g., two, 10 mm diameter culture dishes (Corning Life Sciences, Amsterdam, Holland). Tissue samples, e.g., bone marrow samples or adipose tissue samples, can be kept in culture for a period between about 1 and about 30 days, preferably about 14 days, with periodic half media changes, e.g., every 3-4 days. See Cuthbert, R., et al., Single-platform quality control assay to quantify multipotential stromal cells in bone marrow aspirates prior to bulk manufacture or direct therapeutic use. Cytotherapy (2012); 14(4):431-40, which is incorporated herein by this reference. The colonies formed during the culture period can be visualised for counting using a dye (e.g., methylene blue) and the number of colonies/plate can be manually enumerated, and the number of colonies/ml of bone marrow sample can be calculated.

Blood, Bone Marrow and Lipoaspirate Blood/Saline Fraction Concentration

Blood, bone marrow and lipoaspirate (e.g., lipoaspirate blood/saline fraction) samples can be concentrated by any number of known methods. Blood, bone marrow and plasma concentrating devices are commercially available. In one example, a bone marrow sample or a blood/saline fraction can be concentrated using a concentrator, e.g., Plasmax®Plasma Concentrator or BioCUE® Concentrator (Zimmer Biomet, USA), or other commercially available concentrator. In an example, an aliquot of bone marrow aspirate can be mixed with a proportionate amount of anticoagulant, e.g., acetate citrate dextrose (ACD), and concentrated in a concentrator (e.g., BioCue®) to obtain a bone marrow concentrate. In a preferred example, concentration using Plasmax® Plasma Concentrator or BioCue® results in a fixed volume of bone marrow concentrate or a lipoaspirate concentrate that is approximately 10% of the bone marrow sample or the lipoaspirate blood/saline fraction. For example, 50 ml of bone marrow aspirate can be mixed with 10 ml of ACD, and processed by the BioCUE® device according to the manufacturer's directions. In another example, a volume of 55 ml of blood/saline fraction of lipoaspirate (optionally, mixed with 5 ml ACD) can be processed by the Plasmax® device according to the manufacturer's directions. After centrifugation according to manufacturer's instructions, a layer of concentrated bone marrow or concentrated lipoaspirate fraction can be withdrawn from the concentrator device into a syringe, e.g., an ACD-washed syringe.

An aliquot (e.g., 1 ml) from the bone marrow sample or lipoaspirate sample (blood/saline fraction), pre-concentration and post-concentration, can be analysed for the number of MSCs using the fast enumeration assay described herein. In certain examples, the bone marrow or lipoaspirate concentrate can be left undiluted for staining. In other examples, the bone marrow or lipoaspirate concentrate can be diluted, e.g., 1-fold, 2-fold, 5-fold, 10-fold, or more, before staining. In still other examples, pre-concentration and post-concentration bone marrow samples and lipoaspirate samples can be processed as described above for the LSRII assay and/or the CFU-F assay. In certain examples, platelet counts can be performed for tissue samples pre-concentration and post-concentration using an automated haematopoietic cell counter (e.g., Sysmex; Sysmex Ltd, UK).

Loading MSC-Containing Tissue Samples On Scaffolds

In certain examples, a scaffold can be loaded with a sample of bone marrow aspirate, lipoaspirate, lipoaspirate concentrate or bone marrow concentrate. The yield of MSCs can be determined by an example of the fast enumeration assay before loading the tissue sample to a scaffold, e.g. a collagen scaffold or a hyaluronic scaffold, or commercially available scaffold for facilitating cell growth. Extracellular matrix scaffolds made from adipose tissue are also known and described in Choi, J S et al., Fabrication of porous extracellular matrix scaffolds from human adipose tissue, Tissue Eng. Part C Methods (2010); 16(3):387-96. In an example, a sample of bone marrow aspirate, lipoaspirate, lipoaspirate concentrate, or bone marrow concentrate can be used to load a collagen scaffold, such as Bio-Gide (Geistlich Pharma, Switzerland). The volume of bone marrow aspirate, lipoaspirate, lipoaspirate concentrate, or bone marrow concentrate loaded onto a scaffold can be varied to accommodate varying sizes of scaffold. For example, for a 75 mm³ scaffold piece, approximately 400 μl of bone marrow aspirate, lipoaspirate, lipoaspirate concentrate, or bone marrow concentrate can be loaded onto the scaffold. In some examples, a scaffold can be maintained at 37° C. for a period (e.g., about 2 hours to about 24 hours or more), with gentle mixing, e.g., rocking, to allow MSC attachment to the scaffold.

In one example, the MSCs in the tissue sample can be counted using the fast enumeration assay described herein before loading (pre-loading) the tissue samples onto the scaffold. In other examples, MSCs that have been added to a scaffold but that have not been absorbed on the scaffold can be quantified using the fast enumeration assay described herein (i.e., post-loading enumeration of MSCs remaining in the solution after a period of adsorption). In certain examples, the numbers of MSCs attached to a scaffold can be calculated by subtraction of the numbers of non-adsorbed MSCs (post-loading of the scaffold) from the total quantity of MSCs in the tissue sample (pre-loading of the scaffold). In still other examples, the scaffolds loaded with MSCs can be maintained and cultured for a period, e.g. 1 day to about 60 days, under conditions known in the art, e.g., as described in El-Jawhari, J. J., et al., Collagen-containing scaffolds enhance attachment and proliferation of non-cultured bone marrow multipotential stromal cells. J. Orthop. Res., 2016; 34(4):597-606, which is incorporated herein by this reference. After a desired period in culture, the number of MSCs colonized on the scaffold can be determined. In some examples, scaffolds can be treated to dislodge attached MSCs, such that the MSCs can be extracted and processed for staining and cell counting using the fast enumeration assay described herein. In certain embodiments a collagen scaffold can be digested with collagenase (e.g. for a period of 1 hour) and then the MSCs can be extracted and processed for staining for positive biomarkers of MSCs or negative biomarkers of MSCs, or both, e.g., anti-CD271^{+/high/bright}, and/or anti-CD45^{-/low/dim}.

Statistical Analysis

The statistical analysis and graph preparation can be performed using software, e.g., GraphPad Prism software version 6.0 g. In some examples, the normal distribution of data can be assessed using the Shapiro-Wilk normality test and accordingly the appropriate test for comparative analysis as well as correlation analysis between the data can be applied. In some examples, statistical significance can be shown when p<0.05.

Fast Enumeration Assay Results

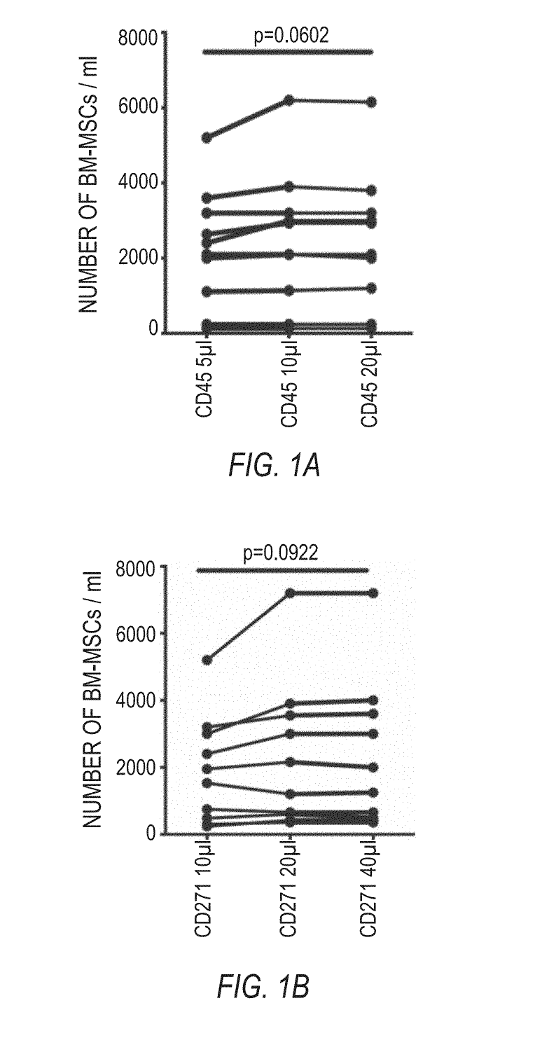

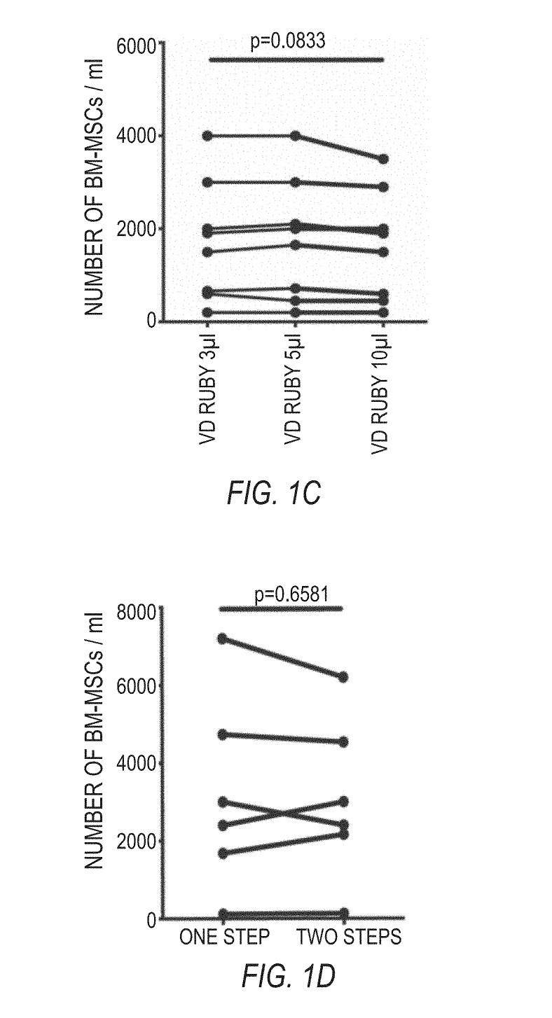

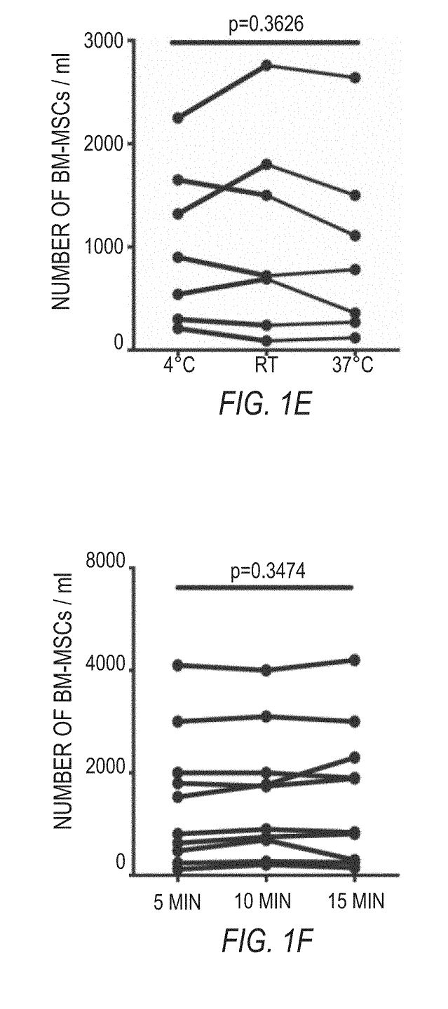

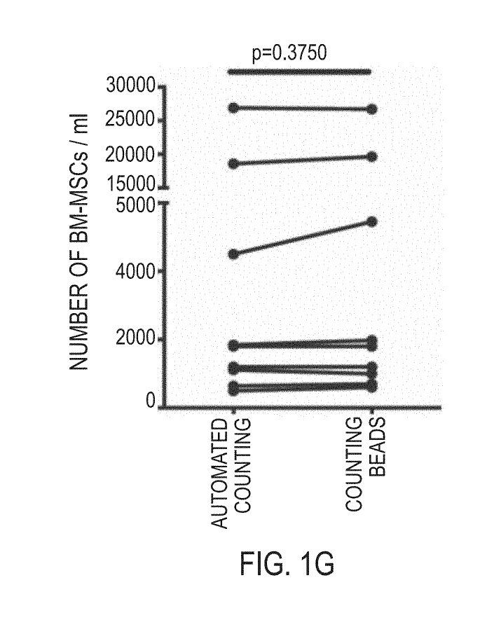

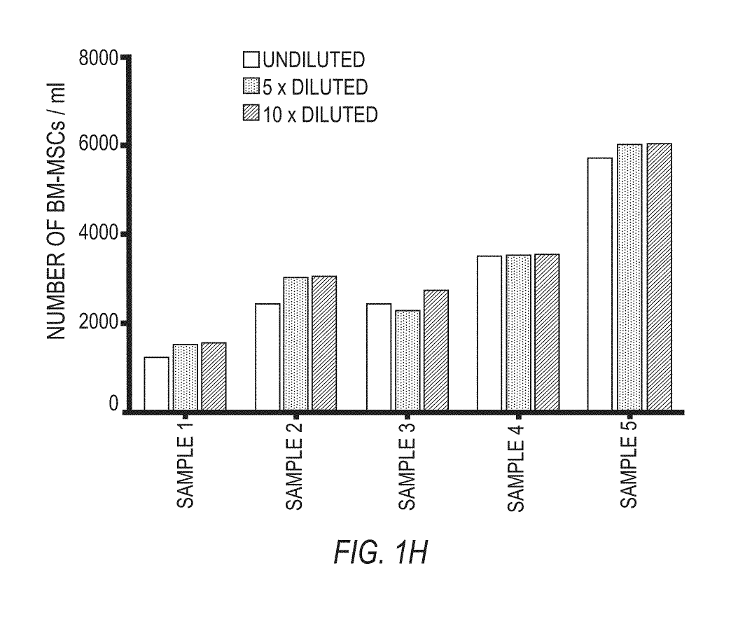

FIGS. 1A through 1H show tissue samples prepared and counted using an example of the fast enumeration assay described herein. MSC numbers counted are shown relative to staining concentrations (FIGS. 1A, 1B, 1C), staining technique (FIG. 1D), staining temperature (FIG. 1E), staining time (FIG. 1F), counting beads (FIG. 1G) and tissue sample dilution (FIG. 1H). In an example, the staining of tissue samples as described herein uses saturating volumes of one or more detecting reagents. In some examples, fluorophore-conjugated antibody volumes can be between about 1 μl and about 100 μl of commercially available reagents can be used, e.g., 1, 3, 5, 10, 15, 20, 35, 30, 35, 40, 45, 50, 55, 60, 65,70, 75, 80, 85, 90, 95 or 100 μl, or any volume therebetween. In certain examples, a saturating volume can comprise about 5 μl, 10 μl, or 20 μl for a tissue sample of about 100 μl. Using such volumes of detecting reagent, the numbers of MSCs using a negative biomarker of MSCs (CD45^{-/low/dim}) can be compared among these staining volumes. As shown in FIG. 1A (sample number=10), using 5 μl of anti-CD45 antibody the number of MSC (CD45^{-/low/dim}) was not statistically different from using 10 μl and 20 μl anti-CD45 antibody (p=0.0602). However, using 10 μl anti-CD45 antibody resulted in counting larger numbers of MSCs compared to 5 μl anti-CD45 antibody in certain samples. In other examples, 10 μl, 20 μl or 40 μl of detecting reagent for a positive biomarker of MSCs (CD271^{+/high/bright}) can be added to about 100 μl of a tissue sample and the number of MSCs can be compared among these staining volumes. As shown in FIG. 1B, the number of MSCs (CD271^{+/high/bright}) were not statistically different (p=0.0922) using different anti-CD271 antibody volumes of 10, 20 and 40 although using 20 μl of anti-CD271 antibody detected more CD271^{+/high/bright} cells than 10 μl anti-CD271 antibody in some samples. The volume of a detecting reagent to detect DNA or nucleated cells, e.g., Vybrant® DyeCycle™ Ruby dye, can be used in volumes from about 1 μl to about 100 μl, e.g., 1, 3, 5, 10, 15, 20, 35, 30, 35, 40, 45, 50, 55, 60, 65,70, 75, 80, 85, 90, 95 or 100 μl, or any volume therebetween. In some examples, 3 μl, 5 μl, or 10 μl of Vybrant® DyeCycle™ Ruby dye can be added to 100 μl of a tissue sample. As shown in FIG. 1C, the numbers of MSCs counted were similar (p=0.0833) using different volumes of dye. In a preferred example, about 10 μl of anti-CD45 antibody can be used to stain about 100 μl of a tissue sample. In another preferred example, about 20 μl of anti-CD271 antibody can be used to stain about 100 μl of a tissue sample. In yet another preferred example, about 3 μl of Vybrant® DyeCycle™ Ruby dye can used to stain about 100 μl of a tissue sample. In another preferred example, 10 μl of anti-CD45 antibody, and 20 μl of anti-CD271 antibody can be used to stain 100 μl of a tissue sample. In yet another preferred example, 3 μl Vybrant® DyeCycle™ Ruby dye can optionally be used to stain 100 μl of a tissue sample that is also stained with 10 μl of anti-CD45 antibody and 20 μl of anti-CD271 antibody.

As described in more detail below, in other examples, a quantity of a dried fluorescent antibody stain or dried dye (e.g., freeze-dried stain or dye) can be combined with a volume of a tissue sample, e.g., 100 μl blood, bone marrow aspirate, lipoaspirate, or a fraction or concentrate of any one or any combination of such tissue samples, such that the volume of tissue sample rehydrates or reconstitutes the stain or dye.

In an example, one or more detecting reagents configured to detect one or more positive biomarkers for MSC (+/high/bright) and one or more detecting reagents configured to detect one or more negative biomarkers for MSC (−/low/dim) can be added to a tissue sample simultaneously. In certain preferred examples, fluorophore-conjugated anti-CD271 antibody for identifying a positive biomarker of MSC (CD271^{+/high/bright}), and fluorophore-conjugated anti-CD45 antibody for identifying a negative biomarker of MSC (CD45^{-/low/dim}), can be added to a tissue sample at the same time; optionally, a live or dead cell detecting reagent, and/or a DNA or nucleated cell detecting reagent, e.g., Vybrant® DyeCycle™ Ruby dye, can also be added to a tissue sample concurrent with the addition of the detecting reagent for the negative biomarker of MSCs (e.g. anti-CD45 antibody) and the addition of the detecting reagent for the positive biomarker of MSC (e.g., anti-CD271 antibody). In one example in which one or more detecting reagents for positive biomarkers for MSC and for negative biomarkers for MSC are added simultaneously to a tissue sample, staining can be performed for 15 minutes or less, at room temperature (15-30° C.). In another example in which one or more detecting reagents for positive biomarkers for MSC and for negative biomarkers for MSC are added to a tissue sample, staining can be performed in a two-step staining method in which one or more detecting reagents are added to the tissue sample and staining is performed for a period of time at a first temperature, and then one or more additional detecting reagents can be added to the tissue sample and the staining can be performed for a period of time at a second temperature that is higher or lower than the first temperature. In any example that can be used in either a one-step staining method or a two-step staining method, staining can be for a period of 1 minute to 30 minutes, e.g., 1, 3, 5, 8, 10, 12, 15, 18, 20, 25, or 30 minutes, or any period therebetween. In a preferred example, staining with detecting reagents can be for a total of about 15 minutes or less. In another preferred example of one-step staining or two-step staining, the staining with detecting reagents can be for a total of 10 minutes or less. In still another preferred example of one-step staining or two-step staining, the staining with one or more detecting reagents can be for a total of 5 minutes or less. In one example, staining with a detecting reagent, e.g., with anti-CD45 antibody and/or anti-CD271 antibody, can be performed for 15 minutes or less at room temperature, followed by staining with a non-antibody detecting reagent, such as a live cell or dead cell dye, or a dye for detecting DNA or nucleated cells (e.g. Vybrant® DyeCycle™ Ruby dye), for an additional period of 15 minutes or less, at 37° C. As shown in FIG. 1D, the numbers of MSCs counted were not significantly different using one-step staining, i.e., detecting agents added concurrently, or two-step staining (p=0.6581).

In another example, a tissue sample can be stained with one-step staining (e.g., simultaneous staining with anti-CD45 antibody, anti-CD271 antibody, and optionally a DNA or a nucleated cell dye e.g., Vybrant® DyeCycle™ Ruby dye) for a total of 15 minutes or less at 4° C., or at room temperature, or at 37° C. As shown in FIG. 1E, MSC numbers counted using different staining temperatures were not significantly different (p=0.3626). In yet another example, staining with all detecting reagents (e.g., simultaneous staining with anti-CD45 antibody, anti-CD271 antibody, and optionally a DNA or nucleated cell dye or live/dead cell dye) can be performed for a total period from 1 minute to about 15 minutes, e.g. 1 minute, 3 minutes, 5 minutes, 10 minutes, 15 minutes, or any time therebetween. As shown in FIG. 1F, the numbers of MSCs counted by the fast enumeration assay (automated counting) were similar (p=0.3474) using staining times of 15 minutes or less. In a preferred example, the staining of tissue sample for MSC enumeration can be performed in a one-step staining using a detecting reagent configured to detect at least one positive biomarker of MSC and detecting reagent configured to detect at least one negative biomarker of MSC, and optionally a DNA or a nucleated cell dye, a cell phase dye, a live cell or a dead cell dye (e.g., simultaneous staining with anti-CD45 antibody, anti-CD271 antibody, and Vybrant® DyeCycle™ Ruby dye) for a period of 5 minutes or less at room temperature (15-30° C.).

In certain examples, data acquisition on the flow cytometer (e.g., Attune®) can include a variety of acquisition variables selected according to the device specifications, e.g., total events counted, events/second, sample flow rate, gating criteria, etc. In one example the time specified to acquire 10,000 events, 25,000 events, 50,000 events, 75,000 events, 100,000 events, 150,000 events, 200,000 events, 250, 00 event, 300,000 events, 350,000 events, 400,000 events, 500,000 events, 550,000 events, 600,000 events, 650,000 events, 700,000 event, 750,000 events, 800,000 events, 850,000 events, 900,000 events, 850,000 events, or 1,000,000 events or more can be selected, or any number of events between about 10,000 and about 1,000,000 events can be selected. In another example, a sample flow rate of 1 ml/min to 20 ml/min can be selected. In a preferred example, acquisition of data can be specified for acquisition within a specified period, e.g., within 1 minute, 5 minutes, 10 minutes, 15 minutes, 20 minutes, 25 minutes, 30 minutes, or any period between about 1 minute and about 30 minutes. In a preferred example, 250,00 events can be selected for acquisition of MSCs in a tissue sample in about 10 minutes or less.

In a preferred example, a tissue sample can be processed using one-step staining at room temperature for 5 minutes or less, using simultaneous fluorescent antibody staining directed against at least one positive biomarker for MSCs (e.g., a biomarker that is positively expressed in MSC, but that is not highly expressed on another cell type in the tissue sample), and fluorescent antibody staining directed against at least one negative biomarker for MSCs (e.g., a biomarker that is positive for another cell type in the tissue sample but that is not highly expressed or is absent in MSCs). In a preferred example, one-step simultaneous staining can be performed for about 5 minutes or less at room temperature (15-30° C.) using fluorescent anti-CD45 antibody, fluorescent anti-CD271 antibody and, optionally, a DNA or nucleated cell dye, e.g., Vybrant® DyeCycle™ Ruby dye such that RBC lysis is not required, and data acquisition can be specified for a total period of 10 minutes or less, providing a fast enumeration assay that can be completed (including staining and acquisition of the specified events) within 15 minutes or less.

To validate the automated counting of MSCs on an automated flow cytometer, an internal control can be provided by adding counting beads of a known concentration to the stained tissue sample. As shown in FIG. 1G, using the fast enumeration assay described herein, both MSC and counting bead enumeration were comparable, confirming the accuracy of flow cytometer automated counting of MSCs (e.g., using Attune®). In yet another example, dilution of tissue samples can be used to test the sensitivity of the fast enumeration assay, i.e., to measure MSC numbers at the lower limit of detection. As shown in FIG. 1H, the dilution of tissue samples demonstrates that the numbers of MSCs counted were consistent when tissue samples were diluted 5-fold (1:5), and 10-fold (1:10), compared to undiluted samples.

In a preferred example, one-step staining of a tissue sample (e.g., 100 μL tissue sample) to detect at least one positive biomarker for MSCs, e.g., using a fluorescent anti-CD27 antibody, and to detect at least one negative biomarker for

MSCs, e.g., using a fluorescent anti-CD45 antibody, and optionally a DNA or nucleated cell dye, or a cell phase or live cell or dead cell dye, for 5 minutes at room temperature, and automated counting acquisition of 250,000 events in 10 minutes or less, as described above, can provide a clinically feasible, fast enumeration assay for MSCs that can accurately determine the number of MSCs (and/or MSC concentration) in a tissue sample within a total assay time of 15 minutes or less.

Fast Enumeration Assay Validation

The fast enumeration assay described herein for fast processing and automated counting of MSCs demonstrates that the percentage of MSCs per total bone marrow cells in a tissue sample can range between 0.001% and 0.07%, with a median of 0.016%. The absolute counts of MSCs provide a median of 1,696 MSCs/ml bone marrow, with a range of 64 to 20,992 MSCs, which is consistent with previously published data and confirms the validity of the fast enumeration assay. See Cuthbert, R., et al., Single-platform quality control assay to quantify multipotential stromal cells in bone marrow aspirates prior to bulk manufacture or direct therapeutic use, Cytotherapy, 2012. 14(4): p. 431-40; and Alvarez-Viejo, M., et al., Quantifying mesenchymal stem cells in the mononuclear cell fraction of bone marrow samples obtained for cell therapy, Transplant Proc, 2013; 45(1): p. 434-9.

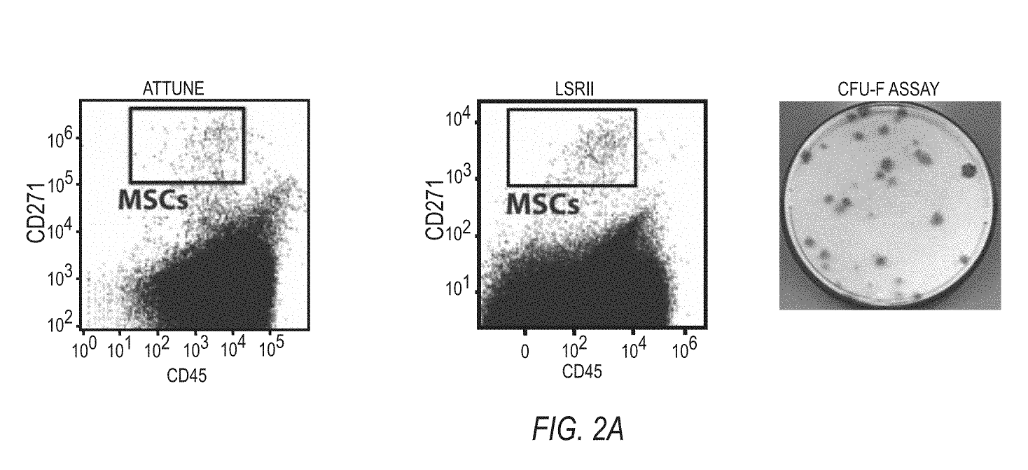

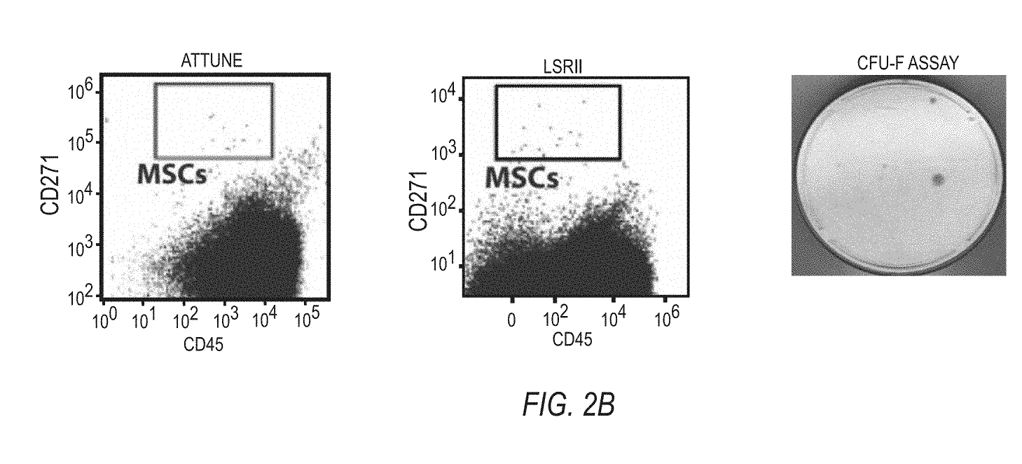

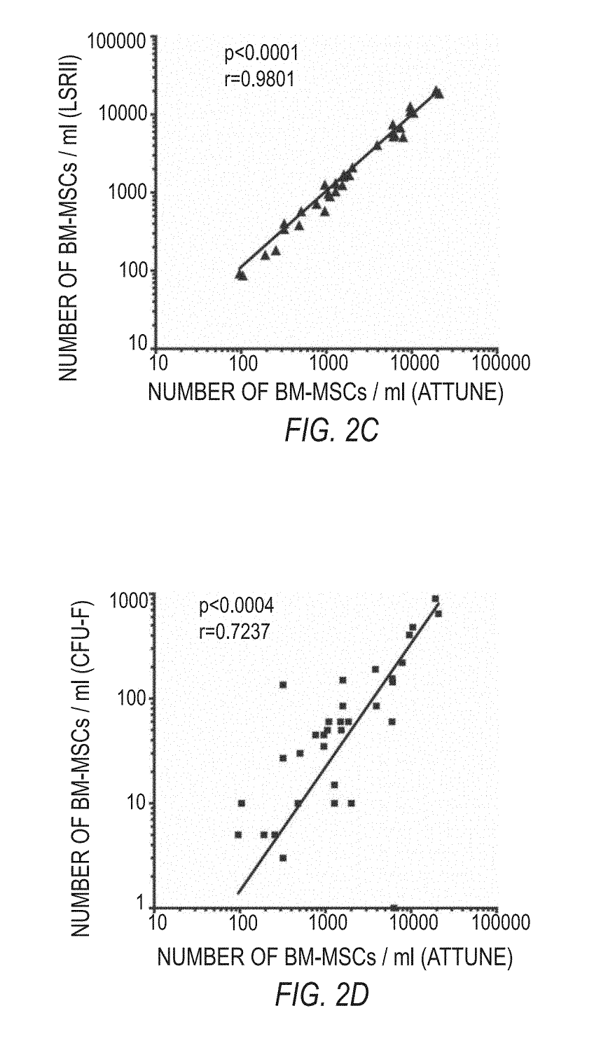

As shown in FIGS. 2A and 2B, when the fast enumeration assay described herein and MSC counts using Attune® are compared with data obtained by automated flow cytometry counts using the LSRII assay (described above) and counted on LSRII flow cytometer (BD Biosciences), and manual counts using CFU-F assay, the results are consistent, i.e. samples with low or high numbers of MSCs are essentially the same in all three assays. The numbers of MSCs obtained from the fast enumeration assay (using Attune®) are similar to numbers counted after preparation using the LSRII assay (median 1,289 MSCs/ml, and range of 58-20,471 MSCs). However, the numbers obtained by the fast enumeration assay were higher than CFU-F colony counts (median 60 colonies/ml bone marrow, range of 4-900 colonies). Nevertheless, as shown in FIGS. 2C and 2D, the numbers of MSCs measured using the fast enumeration assay (using Attune®) significantly correlated with that of the LSRII assay (p<0.0001, r=0.9801) and the CFU-F assay (p=0.0004, r=0.7237).

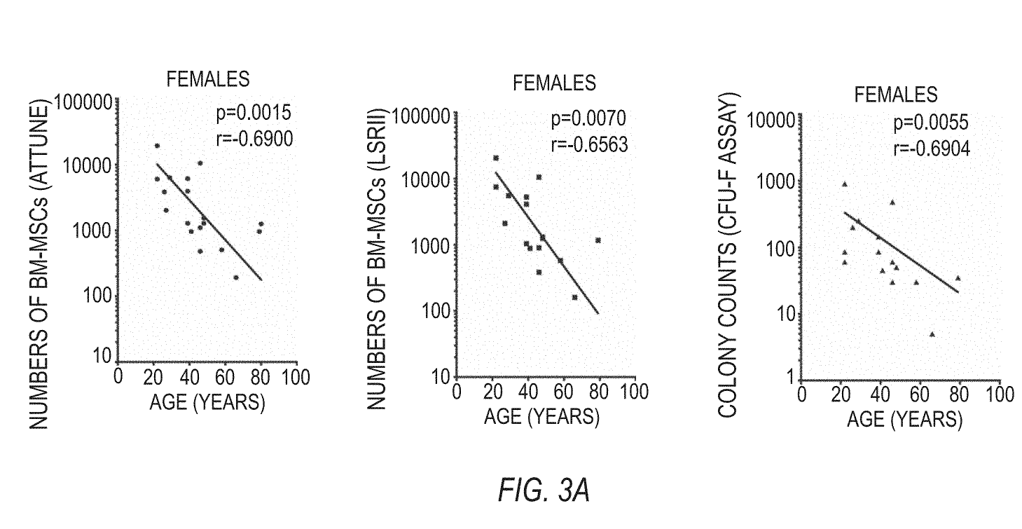

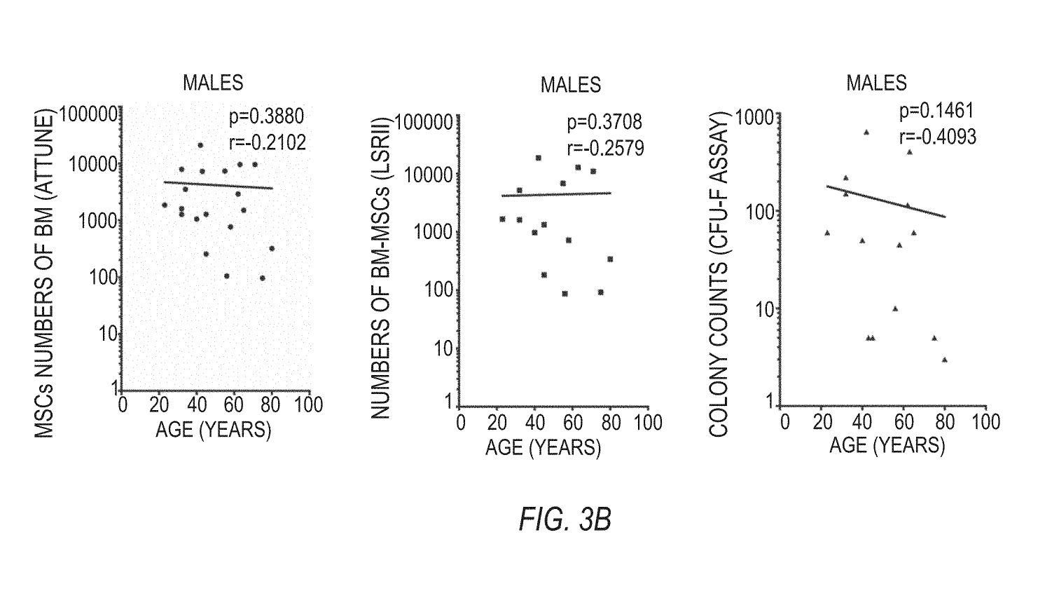

In certain examples, the number of MSCs can be correlated to the age and gender of the bone marrow sample donor. As shown in FIG. 3A, there is a negative correlation between the numbers of MSCs and age in female donors. However, as shown in FIG. 3B, there is not a negative correlation between the number of MSCs and age in the male donors (p=0.3880, r=−0.2102). This correlation was confirmed using the fast enumeration assay (using Attune®)(r=0.6900, p=0.0015), LSRII assay (females: p=0.0070, r=−0.6563; males: p=0.3708, r=0.2577), and CFU-F assay (females: p=0.0055, r=−0.6904; males: p=0.1461, r=0.4093). The correlation data agree with previously published data. See Muschler, G. F., et al., Age- and gender-related changes in the cellularity of human bone marrow and the prevalence of osteoblastic progenitors, J. Orthop. Res. (2001); 19(1): p. 117-25, and Siegel, G., et al., Phenotype, donor age and gender affect function of human bone marrow-derived mesenchymal stromal cells, BMC Med (2013); 11:146. These results demonstrate that the fast enumeration assay (completed in 15 minutes or less) is as good, but much faster than the LSRII assay previously described by Cuthbert et al. (40 minutes), and the CFU-F assay method (14 days).

Fast Enumeration Assay Using Bone Marrow Concentrates and Lipoaspirate Blood/Saline Fraction Concentrates

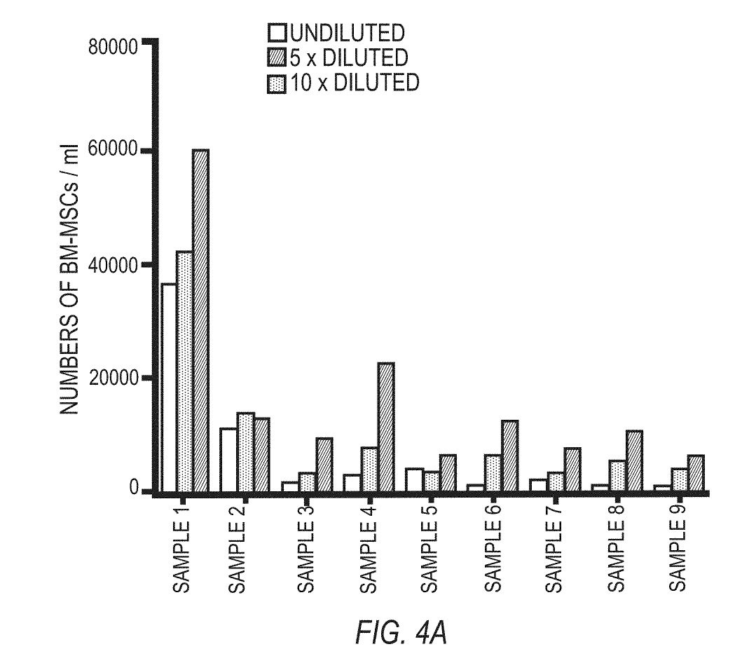

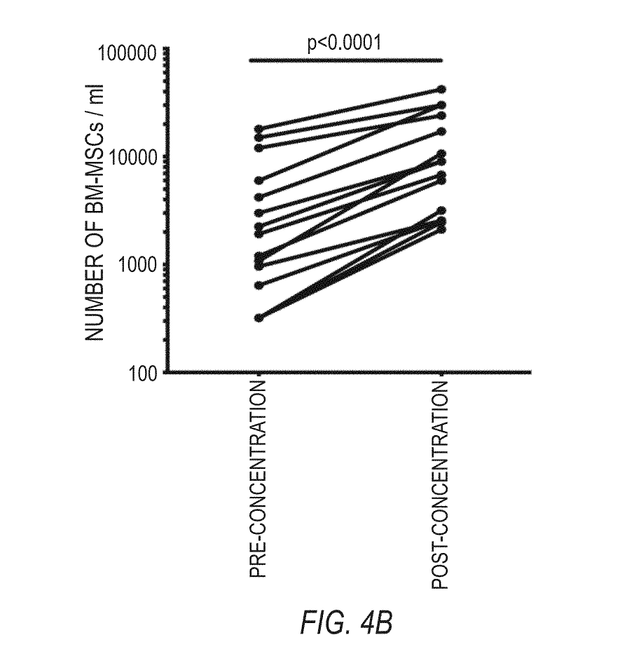

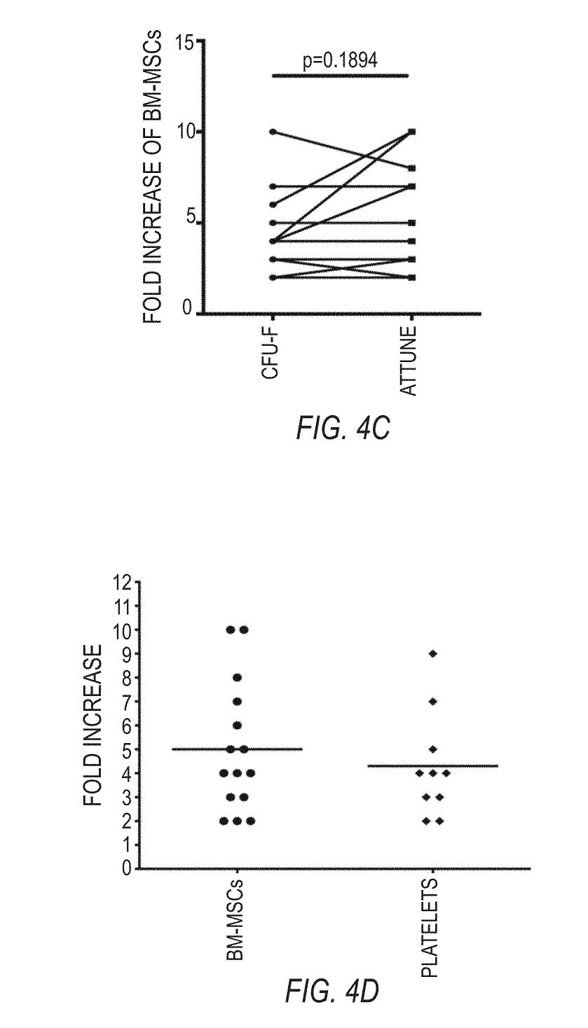

For many therapeutic strategies, including, without limitation, joint regenerative therapy, there is an interest in increasing the number of MSCs delivered by the therapy. Concentrating devices are commercially available and methods are known to concentrate bone marrow aspirates and lipoaspirates (e.g., the blood/saline fraction) using such devices. In one example, a bone marrow aspirate or lipoaspirate sample can be concentrated using the BioCUE® or Plasmax® device (Zimmer Biomet), yielding a fixed volume of concentrate having approximately 10% of the starting tissue sample volume. For example, 60 ml of bone marrow aspirate (with anticoagulant) can be concentrated to an average volume of 6 ml bone marrow concentrate. In another example, the fast enumeration assay described herein can be applied to post-concentration tissue samples. In still other examples, concentrated bone marrow aspirate or concentrated lipoaspirate (blood/saline fraction) can be diluted, e.g., 5-fold (1:5), or 10-fold (1:10), or even greater dilution before processing the diluted bone marrow concentrate or lipoaspirate concentrate using the fast enumeration assay. As shown in FIG. 4A, the quantified MSC numbers were generally higher after 10-fold dilution compared to 5-fold dilution and undiluted samples. In a preferred example, a 10-fold dilution can be performed on a bone marrow concentrate or lipoaspirate concentrate before processing using the fast enumeration assay, to aid efficient staining with detecting reagents and accurate counting of MSCs in the bone marrow concentrate sample. As shown in FIG. 4B, the number of MSCs counted in tissue sample concentrates using the fast enumeration assay can be increased significantly compared to pre-concentration samples (p=0.0001). Furthermore, as shown in FIG. 4C, there is a mean increase of 5-fold in MSCs counted using the fast enumeration assay (Attune®) and CFU-F assay (p=0.1894). In addition, as shown in FIG. 4D, the numbers of platelets in pre-concentration and post-concentration bone marrow samples (platelets counted using Sysmex®) demonstrate a mean increase of 4.2-fold in platelets counts. These data demonstrate the fast enumeration assay can be used to accurately count the MSCs in tissue sample concentrates.

Fast Enumeration Assay of MSCs Attached to a Scaffold

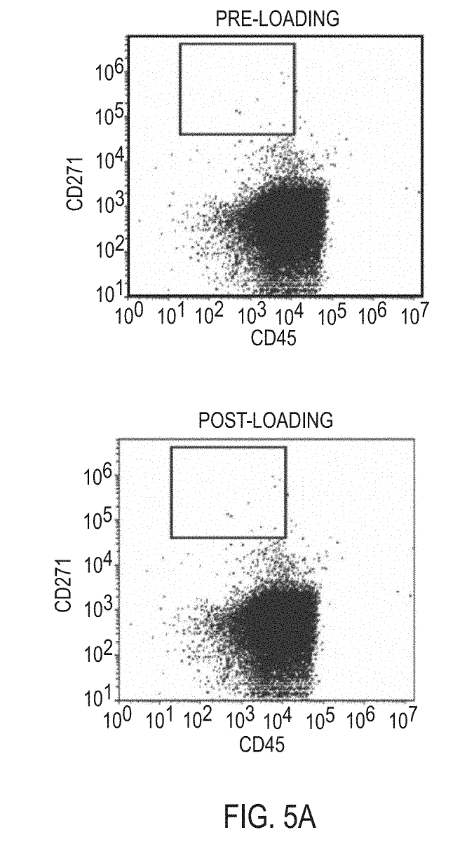

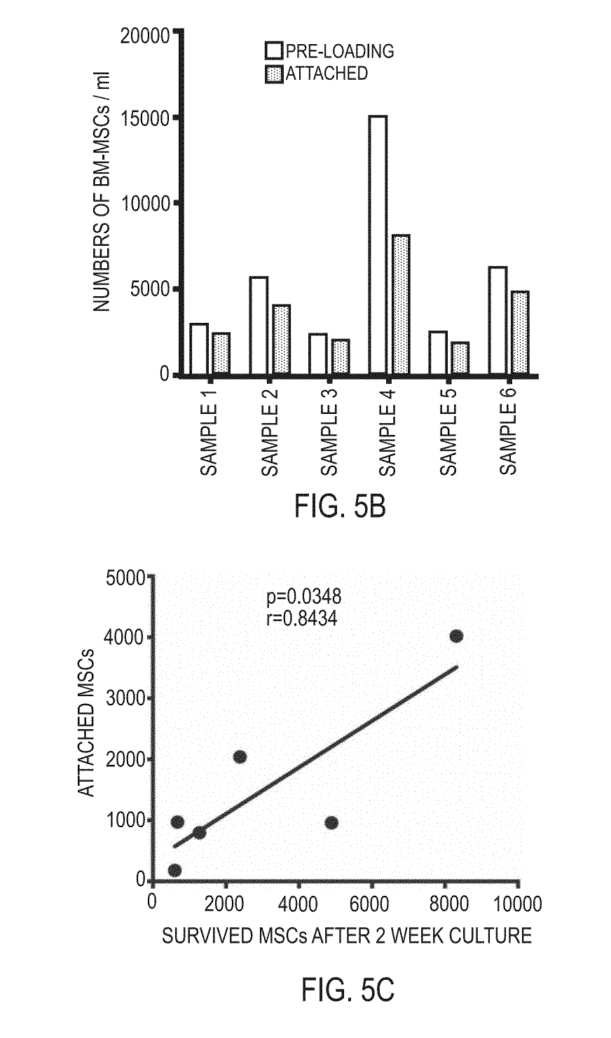

Scaffolds seeded with MSCs, particularly those scaffolds comprising extracellular matrix or collagen, can be of particular importance in orthopaedic therapies, including treating degenerative joint diseases. In some examples, bone marrow aspirates, adipose tissue fractions (including Stromal Vascular Fraction and blood/saline fraction) can be loaded on scaffolds comprising collagen, e.g. BioGide®, or extracellular matrix, or other scaffold material, and the number of attached MSCs can be determined using the fast enumeration assay. In certain examples, as shown in FIG. 5A, enumeration of MSCs can be performed pre-loading of the tissue sample onto a scaffold. In another example, as shown in FIG. 5B, enumeration of MSC can be performed post-loading of the tissue sample onto a scaffold. The numbers of MSCs attached to a scaffold (e.g., Bio-Gide) can vary depending on the pre-loading quantities of MSCs (FIG. 5B). Additionally, MSCs can be released from the digested scaffolds or dislodged from scaffolds following a period of culture of MSCs on the scaffold (e.g., 2 hours to 2 weeks or more). See El-Jawhari, J. J., et al., Collagen-containing scaffolds enhance attachment and proliferation of non-cultured bone marrow multipotential stromal cells, J. Orthop. Res. (2016); 34(4):597-606, which is incorporated herein by this reference. As shown in FIG. 5C, the numbers of MSCs surviving on scaffolds can vary, but strongly correlate with the numbers of attached MSCs (p=0.0348, r=0.8434), confirming the variability of the numbers of both attached MSCs and those colonized on scaffolds and that the fast enumeration assay can effectively detect these differences.

Fast Enumeration Assay Compositions and Kits

In some examples, the reagents of the fast enumeration assay can be premixed for ease of one-step processing. In an example, a composition for quantifying MSCs comprises a detecting reagent configured to detect a positive biomarker of MSCs and a second detecting reagent configured to detect a negative biomarker of MSCs (e.g., a biomarker that is positive for a cell type that is not an MSC and which is absent or of lower expression in MSCs). In certain examples, the composition for quantifying MSCs can further comprise a detecting reagent configured to detect DNA or nucleated cells, live cells or dead cells, determining cell phase. In a particularly preferred example, a composition for enumerating MSCs can comprise a fluorescent anti-CD271 antibody, a fluorescent anti-CD45 antibody, and optionally Vybrant® DyeCycle™ Ruby dye. In another particularly preferred example, a composition for quantifying MSCs can comprise a fluorescent anti-CD271 antibody, a fluorescent anti-CD45 antibody, Vybrant® DyeCycle™ Ruby dye, and counting beads. In any of the foregoing examples, a composition for quantifying MSCs can comprise fluorophore-conjugated antibodies, other dyes (e.g., for discrimination of nucleated cells, live or dead cells, etc.), and optional counting beads, in a dried or a lyophilized form such that a tissue sample or a buffer or water can be added to the composition to rehydrate or reconstitute the lyophilized or dried detecting reagents and other components.

In certain examples, the detecting reagents and optional counting beads can be contained in a standardized tube configured for use in fluorescent activated cell sorting (FACS) and/or other flow cytometry devices. As shown in FIG. 6, an example container (10) for providing the detecting reagents and optional counting beads can comprise a round-bottom cylindrical tube (20) with or without markings (40) e.g., volume markings. In some examples, the cylindrical tube (20) can be configured to be fitted with a cap (30). In certain examples, the cap can be a dual position, snap-fit cap (30). In other examples, a container (10) or a tube (20) can be of a substantially transparent material, e.g., glass or polystyrene. In a preferred example, a tube (20) can be configured to hold about 5 ml or less of a fluid. In still other preferred examples, a tube (20) can have a diameter of approximately 12 mm and a length of approximately 75 mm. In some examples, a porous material (60), e.g., a filter, mesh, or strainer, can be provided with a container (10) to filter a tissue sample to be processed with flow cytometry reagents. In one example, a porous material (60) can be configured to filter a tissue sample. In a preferred example, a porous material (60) can be configured to filter a tissue sample being dispensed into a tube (10) for use in flow cytometry. In one example, a porous material (60) can have pores of any size configured to filter particulate matter from a tissue sample, e.g., a tissue clot or tissue piece. In a preferred example, a porous material can have a pore size ranging from 10 μm to 500 μm or larger, e.g., 10 μm, 15 μm, 20 μm, 25 μm, 30 μm, 35 μm, 40 μm, 45 μm, 50 μm, 55 μm, 60 μm, 65 μm, 70 μm, 75 μm, 80 μm, 85 μm, 90 μm, 95 μm, 100 μm, 125 μm, 150 μm, 175 μm, 200 μm, 225 μm, 250 μm, 275 μm , 300 μm, 325 μm, 350 μm, 375 μm, 400 μm, 425 μm, 450 μm, 475 μm, 500 μm, etc. In other examples, a porous material (60) can optionally be incorporated into a tube (20) or a cap (30), or can be separately provided in a container (10) or can be separately included in a kit for use in flow cytometry methods, and/or for use with a cap (30) or tube (20) suitable for use in flow cytometry methods, including tubes suitable for use in a flow cytometer.

Flow cytometry tubes (20) and caps (30) for use with such tubes are commercially available, e.g., from BD Biosciences, ThermoFisher Scientific, Invitro Technologies, and other sources. In an example, a kit can comprise one or more containers (10), e.g., one or more tubes (20), containing pre-measured amounts of one or more solutions (e.g., 1 μl to about 25 μl, or any amount therebetween), or dried or lyophilized reagents e.g., fluorescent antibodies (50, 51), stains or dyes to detect nucleated, live or dead cells, or cell phase (52), and/or counting beads (53). In one example, a kit can comprise pre-measured amounts of two or more solutions, or two or more dried or lyophilized reagents, or a combination of separate solutions and dried or lyophilized reagents in separate tubes (20) packaged together in a single container (10), e.g., fluorescent antibodies (50, 51), stains or dyes to detect nucleated, live or dead cells, or cell phase (52), and optional counting beads (53). In one example, pre-measured amounts of the solutions, dried or lyophilized reagents, stains or dyes can be configured for storage at room temperature. In a preferred example, a kit can comprise a in a single tube (20) a pre-measured solution (e.g., 1 1 μl to about 25 μl, preferably about 10 to about 20 μl) comprising at least one detecting reagent configured to detect at least one positive biomarker for MSC (50) and at least one detecting reagent configured to detect at least one negative biomarker for MSC (51), and optionally a detecting reagent configured to detect DNA or nucleated cells, or live or dead cells, or cell phase (52), and/or counting beads (53), the solution being cell-free. In another preferred example, a kit can comprise a single tube (20) including at least one pre-measured dried or lyophilized detecting reagent configured to detect at least one positive biomarker for MSC and at least one pre-measured dried or lyophilized detecting reagent configured to detect at least one negative biomarker for MSC (50, 51), and optionally a detecting reagent to detect DNA or nucleated cells, or live or dead cells, or cell phase (52), and/or counting beads (53). In a preferred example, a kit can comprise a pre-measured amount (e.g. 1 μl to 25 μl) of a fluorophore-conjugated antibody configured to detect at least one of CD271, CD29, CD44, CD40a-f, CD51, CD73, CD90, CD105, CD106, CD146, CD166, CD200, and STRO1, and a pre-measured amount (e.g. 1 μl to 25 μl) of a fluorophore-conjugated antibody configured to detect at least one of CD45, CD11b, CD14, CD19, CD31, CD33, CD34, CD79α, and HLA-DR, and a pre-measured amount (e.g. 1 μl to 25 μl) of at least one stain or dye configured to bind DNA or configured to detect a nucleated cell. In order to more conveniently perform the fast enumeration assay described above, compositions for MSC quantification as described in any one of the foregoing examples can be provided in a kit with instructions for use.

The amount of detecting reagent can be provided in a kit or as a composition any amount suitable to provide a saturating volume of the detecting reagent when the tissue sample is added to the detecting reagent, including when the tissue sample (or other liquid) is used to reconstitute a dried or lyophilized detection reagent or composition comprising detecting reagents. Each detecting reagent can be supplied in lyophilized or dried form in tube in any amount between 0.001 μg to 100 μg. In a preferred example, a tube for FACS or flow cytometry, as described above, can be provided with 0.015 μg anti-CD271 antibody (50) (e.g., Miltenyi Biotec, clone ME20.4-1H.4), 1.0 μg anti-CD45 antibody (51) (e.g., BD Biosciences, clone HI30), 2.5 mM nucleated cell dye, e.g., Vybrant DyeCycle Ruby (52) (ThermoFisher Scientific), and optionally counting beads (53) (e.g., CountBright, ThermoFisher Scientific) of a specified number. Counting beads may be provided in any suitable quantity, e.g., 10,000-10,000,000 beads/tube, such that when the staining reagents are reconstituted with a specified volume of tissue sample, buffer or water, the specific concentration of counting beads can be determined.

In a method using the pre-loaded tube (20) having dried, e.g., lyophilized, pre-measured amounts of detecting reagents and optional counting beads, a tissue sample from a donor can be collected as described above. A portion of the tissue sample, e.g., between 10 μl and 1,000 μl, preferably 100 μl, can be added to the pre-loaded tube and gently mixed to suspend or rehydrate the reagents. The tube can be held at room temperature (15° C.-30° C.), preferably in the dark, for 15 minutes or less, preferably 10 minutes or less, and most preferably 5 minutes or less. After the selected period to allow for staining, the tissue sample can optionally be diluted with a buffer (e.g., 2 ml phosphate buffered saline). A flow cytometer can be set for a 10 minute acquisition, with gating for an MSC positive marker (e.g., CD271^{+/high/bright}) and/or an MSC negative marker (e.g. CD45^{-/low/dim}).

In another example, the quantity of MSCs in a tissue sample can be determined using any one of the examples of the fast enumeration assay described above, and the number or concentration of MSCs in the tissue sample can then be used to determine a total quantity of MSCs that is to be provided, or that has been provided, to a subject for regenerative treatment, e.g., treatment of a cartilage or bone defect, bone grafting with autograft or allograft tissues and use as an autologous anti-inflammatory (AAI).

Discussion

The quantity of therapeutic MSCs delivered in a clinical setting, e.g., delivered into degenerative or inflammatory joint, is an important factor that has yet to be optimised for MSC therapies. Although the dose of culture-expanded MSCs can be well-controlled, the production and application of these cells is expensive, labour intensive, requires two steps of surgical procedures, and carries the risk of altered cell function and phenotype. Using blood, blood fractions, bone marrow aspirates, bone marrow concentrates, adipose tissue, lipoaspirates, lipoaspirate fractions, (including blood/saline fraction and Stromal Vascular Fraction) and lipoaspirate concentrates, particularly those loaded on scaffolds, as a source of native MSCs, can save time, effort and cost. The fast enumeration assay described herein provides a clinically feasible, fast and accurate assay for the enumeration of MSCs in bone marrow samples, including bone marrow concentrates and bone marrow samples loaded on scaffolds.

It has been previously shown that CD271 is a specific marker for native MSCs. In contrast, some MSC markers, including integrin molecules (e.g. CD146), are variable according to the topographic location of bone marrow niches, and other markers, such as STRO-1, can be expressed on non-bone marrow cells, e.g., erythroblasts. Existing flow cytometry-based, single-platform methods for counting MSCs require multiple steps of sample processing and a long acquisition time, e.g., on LSRII flow cytometer. Prior studies have shown that CD271⁺ MSCs are also positive for other standard MSC markers, e.g., CD90, CD73 and CD105. According to the inventive methods described herein, using a small volume (100 μl) of a tissue sample, the fast enumeration assay can be completed within 15 minutes or less, including automated cell counting on a compact flow cytometer, such that the methods are feasible for clinical use, including use contemporaneous with therapeutic application of the tissue sample. Multipotential Stromal Cell numbers obtained by the fast enumeration assay can be validated against those counted using existing flow cytometry assays and/or the CFU-F assay. The fast enumeration assay for MSC can be used to determine the total MSC per volume of tissue sample, and thus allow determination of the desired volume of tissue sample required for therapeutic application.

Additionally, the quantification of therapeutically delivered MSCs will be of great value to correlate the MSC dose with clinical response. The fast enumeration assay described herein can aid in the determination of the optimal MSC quantities needed for different regenerative applications, bone grafting with autograft or allograft tissues and use as an autologous anti-inflammatory (AAI). In an example, the fast enumeration assay can be used to determine the number of MSCs administered in a particular clinical application, and the clinician can correlate the clinical response with the number of MSCs administered, and thereby establish a standard for effective therapeutic dose of MSC for the particular application. In an example of a method of using the fast enumeration assay described herein, the assay can be used to verify that a particular tissue sample contains the requisite number of MSC for a given therapeutic application; if the tissue sample contains the required number of MSCs for the application, then the tissue sample can be used directly and without further processing or MSC expansion. In another example, when a specified number of MSC are prescribed for a particular therapeutic application, the fast enumeration assay can be used by the clinician to determine whether a given tissue sample from a donor contains the requisite number of MSC for clinical efficacy; if the tissue sample does not contain the requisite number of MSC for clinical efficacy, then the clinician can use the tissue sample for MSC expansion, further concentrate the tissue sample to obtain the necessary quantity of MSCs for the particular therapeutic application, or obtain and process additional tissue samples to obtain the necessary quantity of MSCs for the particular therapeutic application.

Centrifugation-based approaches for tissue concentration, such as Plasmax® and BioCUE® have been applied to treat cases of femoral AVN with successful outcomes. The results of the fast enumeration assay have demonstrated, in agreement with CFU-F assay, that the number of BM-MSCs can be increased 5-fold using BioCUE®. In addition to MSCs, the platelets were concentrated 4.2-fold using BioCUE®.This shows an additional value of tissue sample concentrates providing more bone and cartilage growth factors, such as Transforming Growth Factor-ß (TGF-ß), Platelet-derived Growth Factor (PDGF) and Vascular Endothelial Growth Factor (VEGF). Although there have been many clinical trials that use bone marrow concentrates, these studies either did not test the number of MSCs in bone marrow concentrates or showed variable results of increased -MSC numbers. Other types of bone marrow concentrators have shown 4-fold and 4.6-fold increases of MSCs. However, another study has shown that two different concentrator devices yielded significantly different numbers of MSCs and different levels of bone marrow growth factors. Collectively, these other studies point to the need for and the clinical value of the fast enumeration assay described herein to determine the MSC yields following bone marrow aspiration and bone marrow concentration.

Scaffolds or matrices have been used in different techniques of joint regenerative therapies, such as microfracture and osteochondral grafts, but their use has been limited due to lack of controlled therapeutic strategy. Bone marrow samples used to load scaffolds, particularly those of collagen or hyaluronic composition, have shown enhanced cartilage repair in OA knee or hip. Additionally, collagen scaffolds loaded with bone marrow concentrate have been proven safe for treatment of focal condylar lesions of knee articular cartilage or talar osteochondral. The fast enumeration assay described herein establishes that the number of MSCs attached to a scaffold are variable depending on initial counts of MSCs in the tissue samples used for loading the scaffold. These differences could explain the dissimilarity of clinical outcomes when loaded scaffolds are used for regenerative therapy. Given the potential value of scaffolds in therapy of various joint degenerative diseases as a structured vehicle for MSC delivery, the fast enumeration assay of the present invention can aid the control and standardization of the quantity of MSCs delivered on these scaffolds.

Applying native MSCs is challenging due to the wide-range variability of MSC numbers between even healthy individuals. Consistent with prior studies, the fast enumeration assay described herein has confirmed a decline in MSC numbers associated with ageing in females, but not males. This differential pattern of MSC numbers implies that the prediction of MSC quantity is very difficult and further highlights the clinical importance of MSC enumeration for MSC therapy.

The clinical improvement of knee osteoarthritis following use of MSC therapy is dose-dependent and 4×10⁸ total nucleated bone marrow cells are considered a threshold for satisfactory clinical scores of pain improvement. Based on that threshold, a median of 0.016% of that count would be 64×10³ MSCs. This number is close to the MSC numbers needed for clinical treatment of fracture non-union. According to the fast enumeration assay described herein, this requisite number of MSCs can be easily obtained from concentration of 60 ml bone marrow aspirate (median of 54×10³ MSCs) using BioCUE®. In contrast to osteoarthritis, the doses of MSCs used for AVN therapy have been reported to vary ranging from 14.7×10⁴ to 92×10⁷ (as counted by CFU-F assays), probably because of the different numbers of participants in these studies. Critically, the fast enumeration assay disclosed herein can help to standardize the therapeutic dose of MSCs for AVN treatment by correlation of MSC doses with the lesion size and the levels of clinical response.

The above detailed description includes references to the accompanying drawings, which form a part of the detailed description. The drawings show, by way of illustration, specific embodiments in which the invention can be practiced. These embodiments are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

Method examples described herein can be machine or computer-implemented at least in part. Some examples can include a computer-readable medium or machine-readable medium encoded with instructions operable to configure an electronic device to perform methods as described in the above examples. An implementation of such methods can include code, such as microcode, assembly language code, a higher-level language code, or the like. Such code can include computer readable instructions for performing various methods. The code may form portions of computer program products. Further, in an example, the code can be tangibly stored on one or more volatile, non-transitory, or non-volatile tangible computer-readable media, such as during execution or at other times. Examples of these tangible computer-readable media can include, but are not limited to, hard disks, removable magnetic disks, removable optical disks (e.g., compact disks and digital video disks), magnetic cassettes, memory cards or sticks, random access memories (RAMs), read only memories (ROMs), and the like.

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to comply with 37 C.F.R. § 1.72(b), to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed example. Thus, the following claims are hereby incorporated into the Detailed Description as examples or embodiments, with each claim standing on its own as a separate embodiment, and it is contemplated that such embodiments can be combined with each other in various combinations or permutations. The scope of the invention should be determined with reference to the appended claims, along with the full scope of equivalents to which such claims are entitled. In the event of inconsistent usages between this document and any documents incorporated herein by reference, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim.

Although the terms first, second, third, etc. may be used herein to describe various elements, components, regions, layers and/or sections, these elements, components, regions, layers and/or sections should not be limited by these terms. These terms may be only used to distinguish one element, component, region, layer or section from another region, layer or section. Terms such as “first,” “second,” and other numerical terms when used herein do not imply a sequence or order unless clearly indicated by the context. Thus, a first element, component, region, layer or section discussed below could be termed a second element, component, region, layer or section without departing from the teachings of the example embodiments. Moreover, in the following claims, the terms “first,” “second,” and “third,” etc. are used merely as labels, and are not intended to impose numerical requirements on their objects

The terminology used herein is for the purpose of describing particular example embodiments only and is not intended to be limiting. As used herein, the singular forms “a,” “an,” and “the” may be intended to include the plural forms as well, unless the context clearly indicates otherwise. The terms “comprises,” “comprising,” “including,” and “having,” are inclusive and therefore specify the presence of stated features, integers, steps, operations, elements, and/or components, but do not preclude the presence or addition of one or more other features, integers, steps, operations, elements, components, and/or groups thereof. The method steps, processes, and operations described herein are not to be construed as necessarily requiring their performance in the particular order discussed or illustrated, unless specifically identified as an order of performance. It is also to be understood that additional or alternative steps may be employed.

The foregoing description of the embodiments has been provided for purposes of illustration and description. It is not intended to be exhaustive or to limit the disclosure. Individual elements or features of a particular embodiment are generally not limited to that particular embodiment, but, where applicable, are interchangeable and can be used in a selected embodiment, even if not specifically shown or described. The same may also be varied in many ways. Such variations are not to be regarded as a departure from the disclosure, and all such modifications are intended to be included within the scope of the disclosure.

What is claimed is:

1. A method of quantifying multipotent stromal cells (MSCs) in a tissue sample, comprising:
contacting a tissue sample with a first detecting reagent configured to detect to a positive cellular marker that is highly expressed in MSC, and a second detecting reagent configured to detect a negative cellular marker that is absent or has low expression in MSCs; and
counting cells in the tissue sample using flow cytometry, wherein the contacting and counting are completed in 20 minutes or less.

2. The method of claim 1, wherein red blood cells in the tissue sample are not removed or lysed before counting the cells.

3. The method of claim 1, wherein the positive cellular marker comprises CD271, CD29, CD44, CD40a-f, CD51, CD73, CD90, CD105, CD106, CD146, CD166, CD200, STRO1, or a combination thereof.

4. The method of claim 1, wherein the negative cellular marker comprises CD45, CD11b, CD14, CD19, CD31, CD33, CD34, CD79α, HLA-DR, or a combination thereof.

5. The method of claim 1, wherein the contacting further includes contacting the tissue sample with a third detecting reagent configured to only detect cells having DNA or a nucleus.

6. The method of claim 1, wherein the negative cellular marker is highly expressed in another cell type present in the tissue sample.

7. The method of claim 1, wherein the contacting and counting are completed in about 15 minutes or less.

8. The method of claim 1, wherein the flow cytometer is configured such that the specified number of acquisition events are obtained in about 10 minutes or less.

9. The method of claim 1, further comprising administering the tissue sample to a subject.

10. A kit for quantifying multipotential stromal cells (MSCs) comprising:
a first reagent configured to detect a positive cellular marker that is highly expressed in MSCs;
a second reagent configured to detect a negative cellular marker that is absent or weakly expressed in MSCs but that is highly expressed in another cell type in blood, bone marrow, or adipose tissue; and
a third reagent configured to detect nucleated cells;
wherein the first reagent, second reagent, and third reagent are packaged together.

11. The composition of claim 10, wherein the positive cellular marker comprises CD271, CD29, CD44, CD40a-f, CD51, CD73, CD105, CD106, CD146, CD166, CD200, STRO1, or a combination thereof.

12. The kit of claim 10, wherein the negative cellular marker comprises CD45, CD11b, CD14, CD19, CD31, CD33, CD34, CD79α, HLA-DR, or a combination thereof.

13. The kit of claim 10, further comprising counting beads.

14. The kit of claim 10, wherein the first reagent, the second reagent, and the third reagent are provided in a dried form.

15. The kit of claim 10, wherein the first reagent, the second reagent and the third reagent are premixed.

16. The kit of claim 10, wherein the package comprises a tube configured for use in a flow cytometer.

17. The kit of claim 10, wherein the first reagent, the second reagent and the third reagent are each provided in amount sufficient to saturate a 100 μl tissue sample.

18. A method for regenerative therapy using multipotential stromal cells (MSCs), the method comprising:
contacting a portion of a tissue sample with a first reagent configured to identify a first marker that is highly expressed in MSCs, a second reagent configured to identify a second marker that is absent or weakly expressed in MSC but is highly expressed in another cell type in the tissue sample, and a third reagent configured to identify nucleated cells in the tissue sample;
quantifying the number of MSCs in the tissue sample or the concentration of MSCs in the tissue sample using flow cytometry; and
administering the tissue sample to a subject.

19. The method of claim 18, wherein the quantifying is performed proximal to the time of administering the tissue sample to the subject.

20. The method of claim 18, wherein the tissue sample is incubated with a scaffold, and the method further comprises determining the number of MSCs adsorbed onto the scaffold.
